(19) United States
(12) Patent Application Publication
(10) Pub. No.: US 2020/0347445 A1
(43) Pub. Date: Nov. 5, 2020

(54) METHOD OF RAPIDLY DETECTING THE PRESENCE OF NUCLEIC ACID TARGET MOLECULES

(71) Applicant: ADOR DIAGNOSTICS S.R.L, Rome (IT)

(72) Inventors: Vladimir HURGIN, Gan Yavne (IL); Nives HODKO, Poway, CA (US); Dalibor HODKO, Poway, CA (US); Zuxu YAO, San Diego, CA (US)

(73) Assignee: ADOR DIAGNOSTICS S.R.L, Rome (IT)

(21) Appl. No.: 16/958,403

(22) PCT Filed: Dec. 27, 2018
(86) PCT No.: PCT/IL2018/051400

§ 371 Date: Jun. 26, 2020

Related U.S. Application Data
(60) Provisional application No. 62/610,997, filed on Dec. 28, 2017.

(51) Int. Cl. C12Q 1/6837

(52) U.S. Cl. CPC C12Q 1/6837

(57) ABSTRACT

A room-temperature shelf-storable electrophoretic array for use in a method of rapidly detecting the presence of at least one nucleic acid target molecule, from among a multiplicity of pre-selected nucleic acid target molecules, in a solution, the electrophoretic array including a multiplicity of immobilized, mutually spaced and mutually electrically separated microgel deposits, each of the multiplicity of immobilized mutually spaced and mutually electrically separated microgel deposits containing materials suitable for performing rolling circle amplification and binding of at least one of the multiplicity of pre-selected nucleic acid target molecules, each of the microgel deposits containing at least the following elements pre-anchored therein: an RCA probe specific to of at least one of the multiplicity of pre-selected nucleic acid target molecules and at least one primer.

### REFERENCE TO RELATED APPLICATIONS

Reference is now made to WO2018/122856, published Jul. 5, 2018, WO 2018/122852, published Jul. 5, 2018 and PCT/IL2018/050726, filed Jul. 4, 2018, which are believed to be related to the present application, the disclosures of which are hereby incorporated by reference.

Reference is also hereby made to U.S. Provisional Patent Application Ser. No. 62/610,997, filed Dec. 28, 2017, the disclosure of which is hereby incorporated by reference and priority of which is hereby claimed.

### FIELD OF THE INVENTION

The present invention generally relates to rolling-circle amplification.

### SUMMARY OF THE INVENTION

The present invention seeks to provide improved methods of rolling-circle amplification (RCA).

There is thus provided in accordance with a preferred embodiment of the present invention a room-temperature shelf-storable electrophoretic array for use in a method of rapidly detecting Che presence cf at least one nucleic acid target molecule, from among a multiplicity of pre-selected nucleic acid target molecules, in a solution, the room-temperature shelf-storable electrophoretic array including a multiplicity of immobilized, mutually spaced and mutually electrically separated microgel deposits, each of the multiplicity of immobilized, mutually spaced and mutually electrically separated microgel deposits containing materials suitable for performing rolling circle amplification and binding of at least one of the multiplicity of pre-selected nucleic acid target molecules, each of the microgel deposits containing at least the following elements pre-anchored therein: an RCA probe specific to of at least one of the multiplicity of pre-selected nucleic acid target molecules and at least one primer.

Preferably, the microgel deposits are dehydrated and are rehydratable when exposed to a solution containing at least one nucleic acid target molecule. Additionally or alternatively, the at least one printer includes at least one forward primer and at least one reverse primer.

In accordance with a preferred embodiment of the present invention the RCA probe is pro-hybridized to the at least one primer.

In accordance with a preferred embodiment of the present invention each of the microgel deposits when hydrated has a generally hemispherical shaped configuration.

In accordance with a preferred embodiment of the present invention the multiplicity of immobilized, mutually spaced and mutually electrically separated microgel deposits define a corresponding multiplicity of immobilized, mutually spaced and mutually electrically separated microgel regions and the electrophoretic array is employed in carrying out a method including introducing the solution to each of the multiplicity of immobilized, mutually spaced and mutually electrically separated microgel regions, performing rolling circle amplification at least generally simultaneously at each of the multiplicity of immobilized, mutually spaced and mutually electrically separated microgel regions, while applying electric fields thereto during various stages of the rolling circle amplification and detecting the presence of at least one of the multiplicity of pre-selected nucleic acid target molecules at at least one corresponding one of the immobilized, mutually spaced and mutually electrically separated microgel regions, wherein the detecting occurs within a short time period of the introducing, the short time period being less than 30 minutes.

There is also provided in accordance with another preferred embodiment of the present invention a method of rapidly detecting the presence of at least one nucleic acid target molecule, from among a multiplicity of pre selected nucleic acid target molecules, in a solution, the method including introducing the solution to at least a multiplicity of immobilized, mutually spaced and mutually electrically separated microgel regions on an electrophoretic array, each of the multiplicity of immobilized, mutually spaced and mutually electrically separated microgel regions containing a microgel deposit containing materials suitable for binding of a different one of the multiplicity of pre-selected nucleic acid target molecules and performing rolling circle amplification, performing rolling circle amplification at least generally simultaneously at the immobilized, mutually spaced and mutually electrically separated microgel regions, while applying electric fields thereto during various stages of the rolling circle amplification and detecting the presence of at least one of the multiplicity of pre-selected nucleic acid target molecules at least one corresponding one of the immobilized, mutually spaced and mutually electrically separated microgel regions, wherein the detecting occurs within a short lime period of the introducing, the short time period being less than 30 minutes.

In accordance with a preferred embodiment of the present invention the detecting includes optical detection. Preferably, the detecting includes fluorescence detection.

In accordance with a preferred embodiment of the present invention the applying electric fields thereto occurs during at least two different stages in the rolling circle amplification.

Preferably, the electric fields are at least generally the same at each of the immobilized, mutually spaced and mutually electrically separated microgel regions. In accordance with a preferred embodiment of the present invention the detecting occurs within a time duration of less than 20 minutes. More preferably, the detecting occurs within a time duration of less than 15 minutes.

In accordance with a preferred embodiment of the present invention the applying electric fields during the rolling circle amplification includes at least one of the following: applying an electric field to the immobilized, mutually spaced and mutually electrically separated microgel regions for driving nucleic acid target molecules in the solution to the microgel deposits, applying an electric field to the immobilized, mutually spaced and mutually electrically separated microgel regions for driving nucleic acid target molecule-RCA probe hybridization products in the solution to the microgel deposits, applying an electric field to the immobilized, mutually spaced and mutually electrically separated microgel regions for recapturing RCA amplicons that drift away from the microgel deposits, applying an electric field to the immobilized, mutually spaced and mutually electrically separated microgel regions for driving RCA probes into the microgel deposits for hybridization with at least one of capture probes and primers already bound to the microgel deposits, applying an electric field to the immobilized, mutually spaced and mutually electrically separated microgel regions for removing undesired molecules from the microgel regions, applying an electric field to the immobilized, mutually spaced and mutually electrically separated microgel regions for stretching RCA amplicons that are bound to the microgel deposits, applying an electric field to the immobilized, mutually spaced and mutually electrically separated microgel regions for compressing RCA amplicons that are bound to the microgel deposits, applying an electric field to the immobilized, mutually spaced and mutually electrically separated microgel regions for stirring RCA reagents in the vicinity of RCA amplicons that are bound to the microgel deposits, applying an electric field to the immobilized, mutually spaced and mutually electrically separated microgel regions for enhancing the speed of enzyme activity in RCA and applying electric field of sequentially reversing polarity to the immobilized, mutually spaced and mutually electrically separated microgel regions for enhancing stringency of binding of RCA amplicons to the microgel deposits.

In accordance with a preferred embodiment of the present invention the electrophoretic array includes a room-temperature shelf-storable electrophoretic array.

Preferably, the electrophoretic array includes a multiplicity of immobilized, mutually spaced and mutually electrically separated microgel deposits, each of the multiplicity of immobilized, mutually spaced and mutually electrically separated microgel deposits containing materials suitable for performing rolling circle amplification and binding of at least one of the multiplicity of pre-selected nucleic acid target molecules, each of the microgel deposits containing at least the following elements pre-anchored therein: an RCA probe specific to of at least one of the multiplicity of pre-selected nucleic acid target molecules and at least one primer.

### BRIEF DESCRIPTION OF THE DRAWINGS

The present invention will be understood and appreciated more fully from the following detailed description, taken in conjunction with the drawings in which:

FIGS. 1A and 1B are simplified pictorial assembled and exploded view illustrations of an electrophoretic array assembly constructed and operative in accordance with a preferred embodiment of the invention including a multiplicity of immobilized, mutually spaced and mutually electrically separated microgel regions during rolling-circle amplification (RCA) operation:

FIGS. 2A and 2B are simplified pictorial assembled and exploded view illustrations of the electrophoretic array assembly of FIGS. 1A and 1B including a multiplicity of immobilized, mutually spaced and mutually electrically separated microgel regions in a dehydrated storage operative orientation:

FIGS. 3A, 3B, 3C, 3D, 3E, 3F, 3G, 3H and 3I are together a simplified illustration of a preferred method of producing the electrophoretic array employed in the embodiment of FIGS. 1A-2B;

FIGS. 4A, 4B, 4C, 4D, 4E, 4F, 4G, 4H, 4I and 4J are simplified illustrations of typical steps in rapid detection of the presence of at least one nucleic acid target molecule in accordance with one embodiment of the present invention;

FIGS. 5A, 5B, 5C, 5D, 5E, 5F, 5G, 5H, 5I and 5J are simplified illustrations of typical steps in rapid detection of the presence of at least one nucleic acid target molecule in accordance with one embodiment of the present invention;

FIGS. 6A, 6B, 6C, 6D, 6E, 6F, 6G, 6H, 6I and 6J are simplified illustrations of typical steps in rapid detection of the presence of at least one nucleic acid target molecule in accordance with one embodiment of the present invention;

FIGS. 7A, 7B, 7C, 7D, 7E, 7F, 7G, 7H, 7I and 7J are simplified illustrations of typical steps in rapid detection of the presence of at least one nucleic acid target molecule in accordance with one embodiment of the present invention;

FIG. 8 is a diagram summarizing the results of Example I; and

FIG. 9 is a diagram summarizing the results of Example II.

### DETAILED DESCRIPTION OF PREFERRED EMBODIMENTS

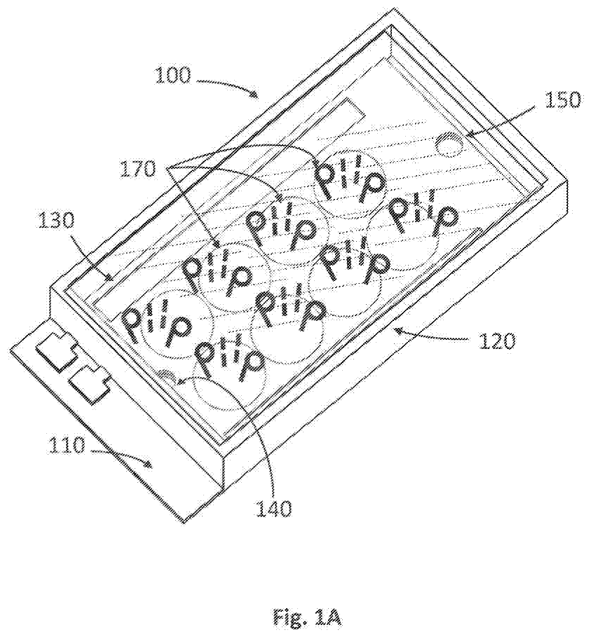

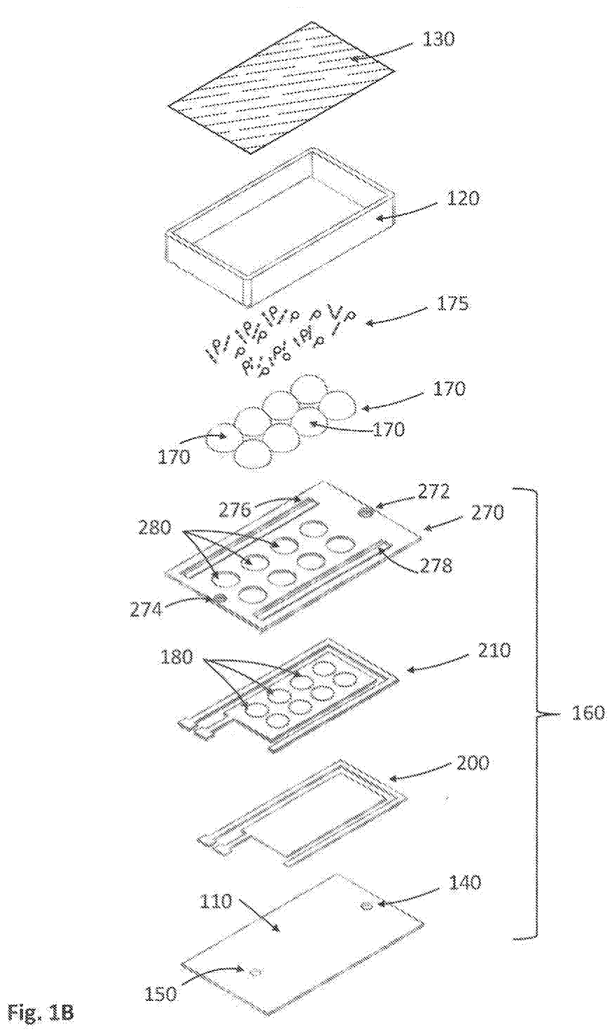

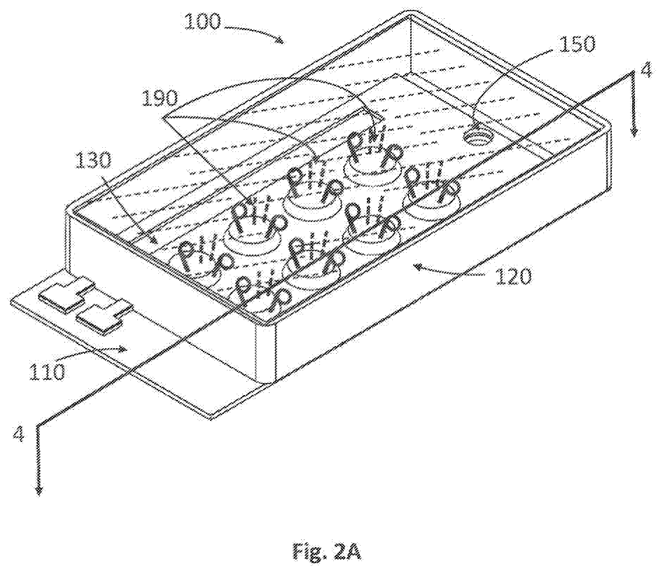

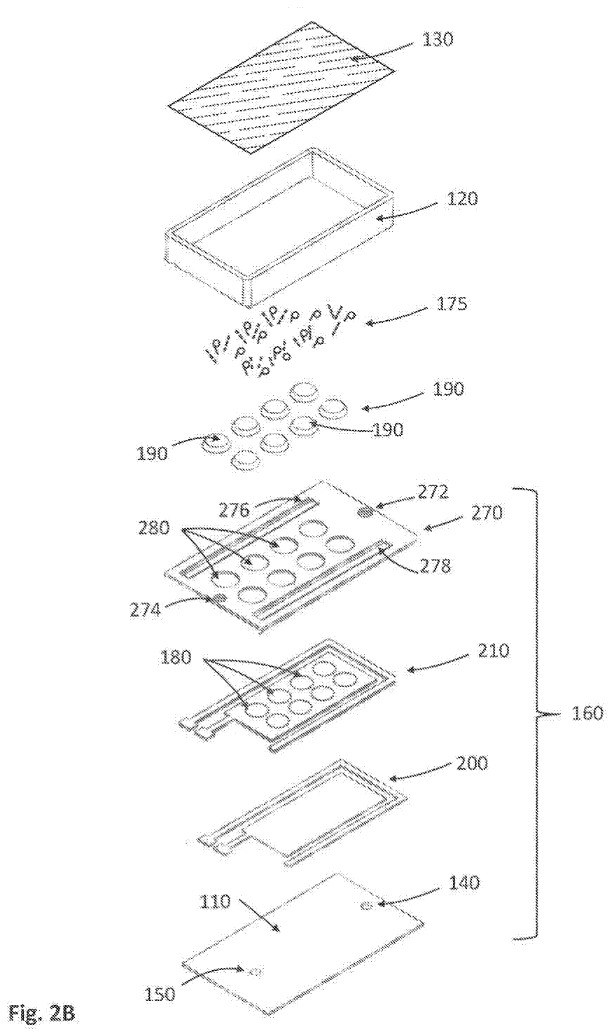

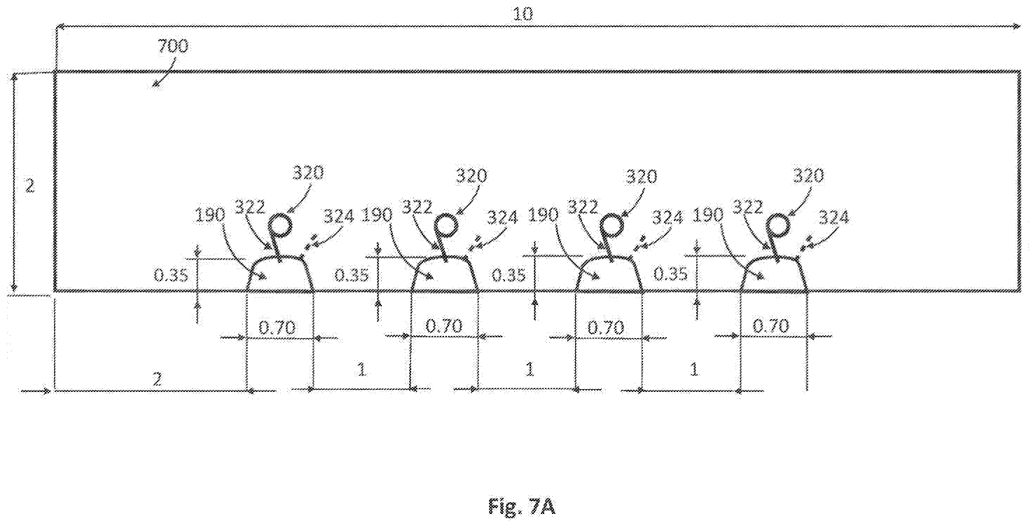

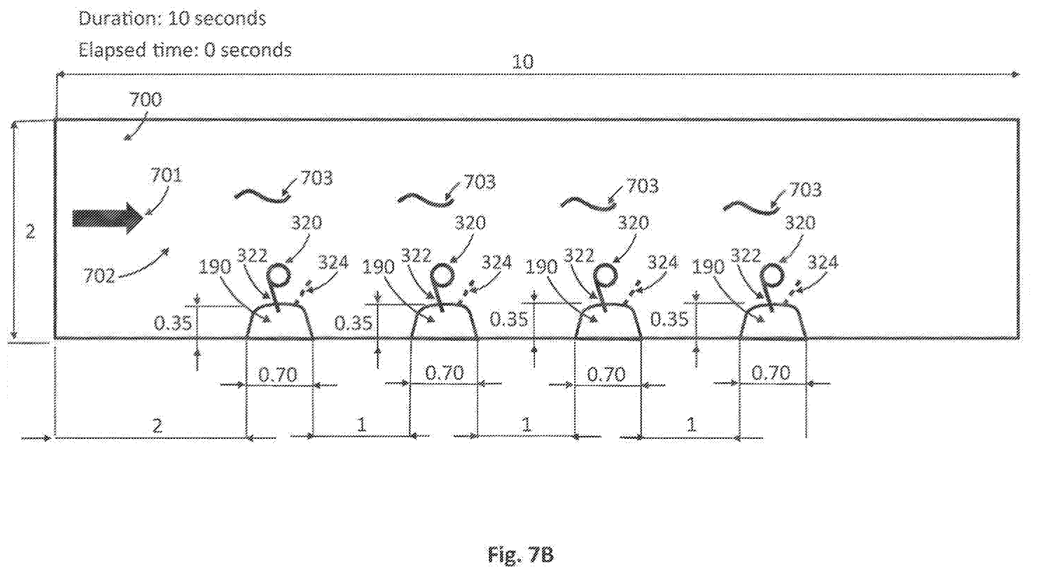

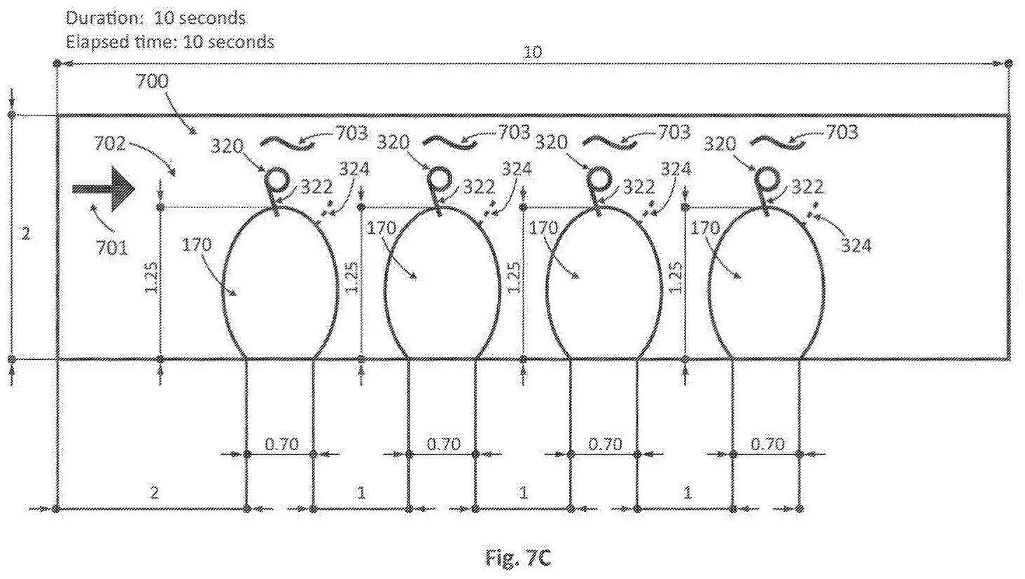

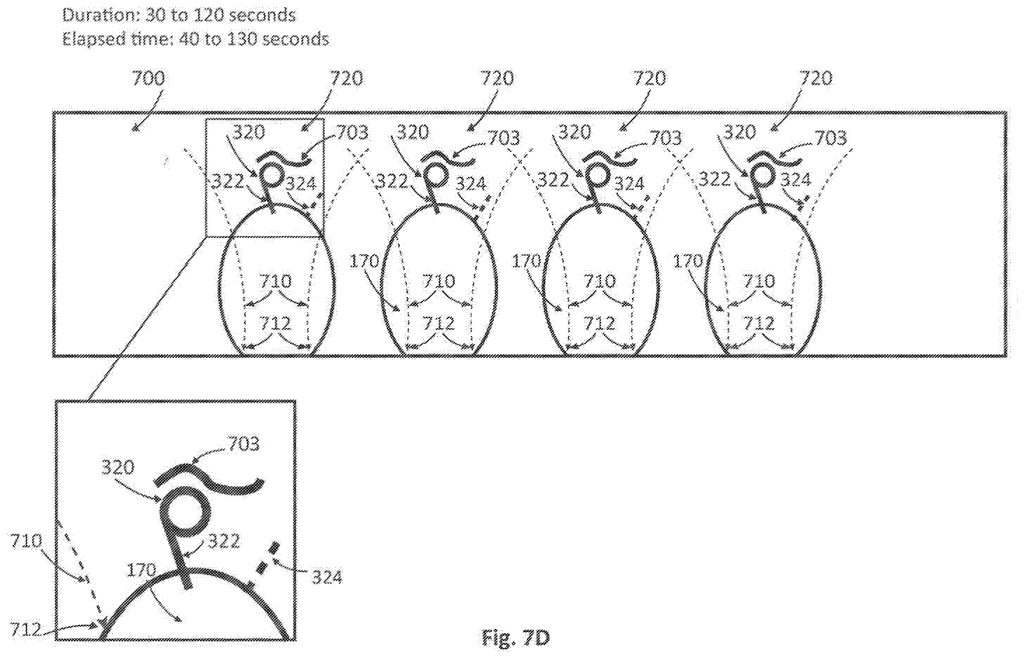

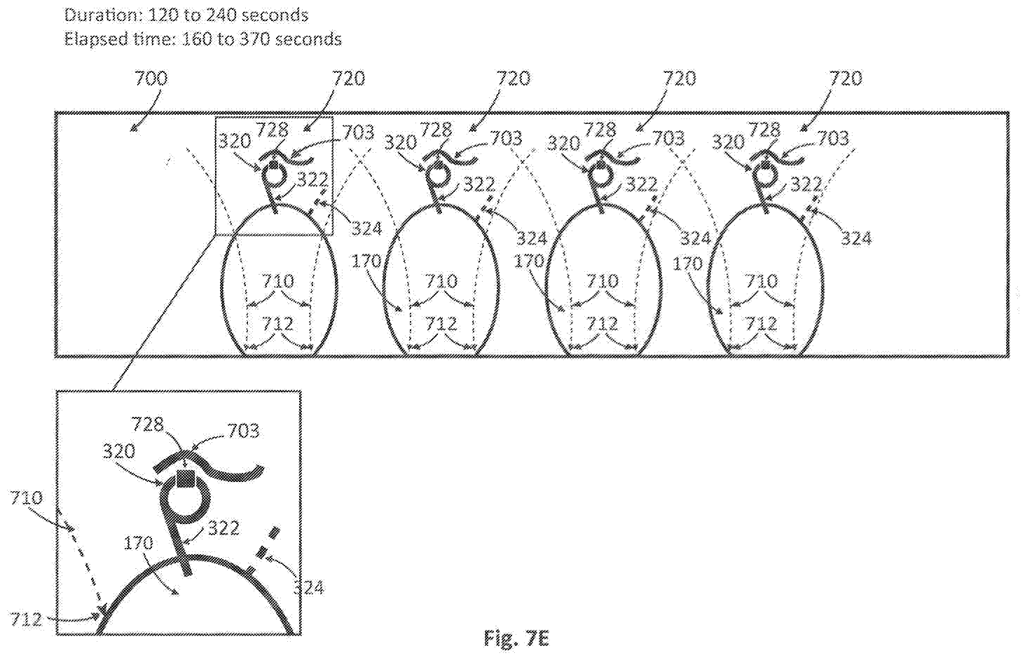

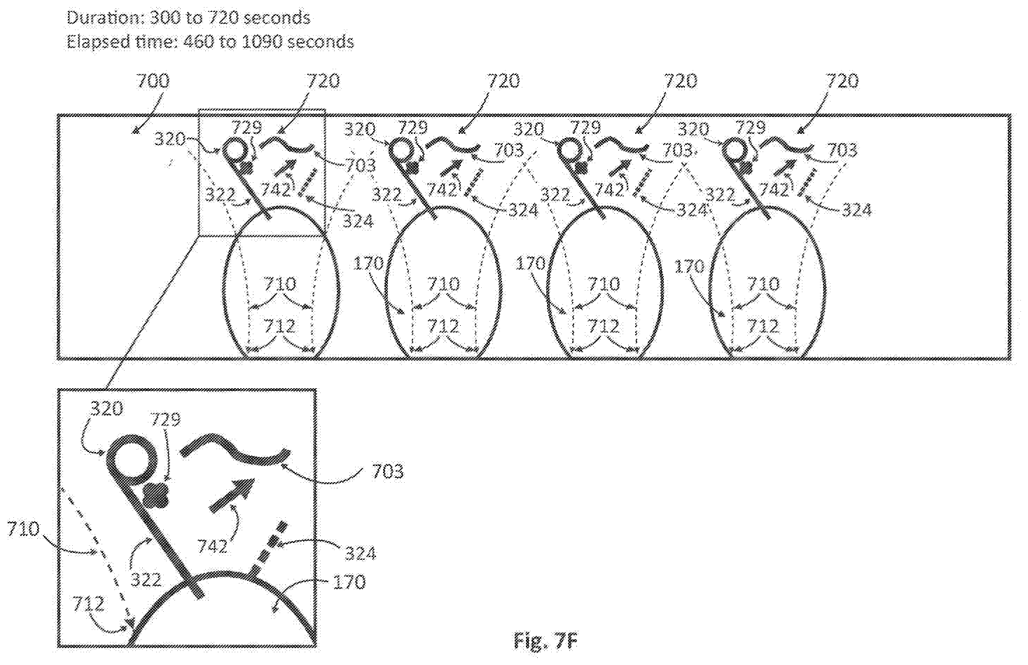

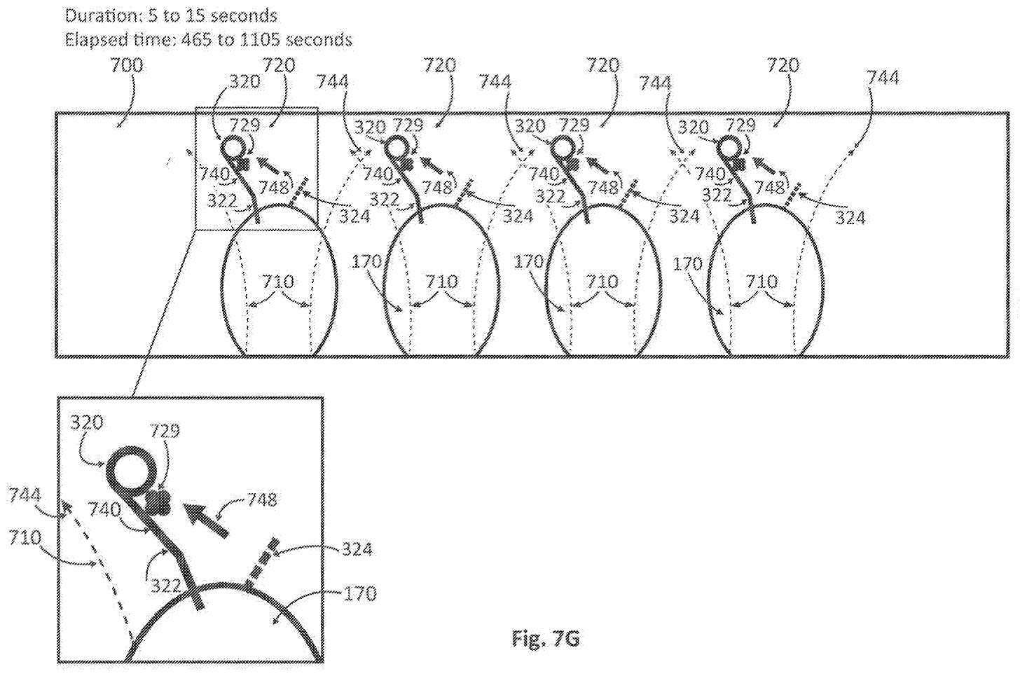

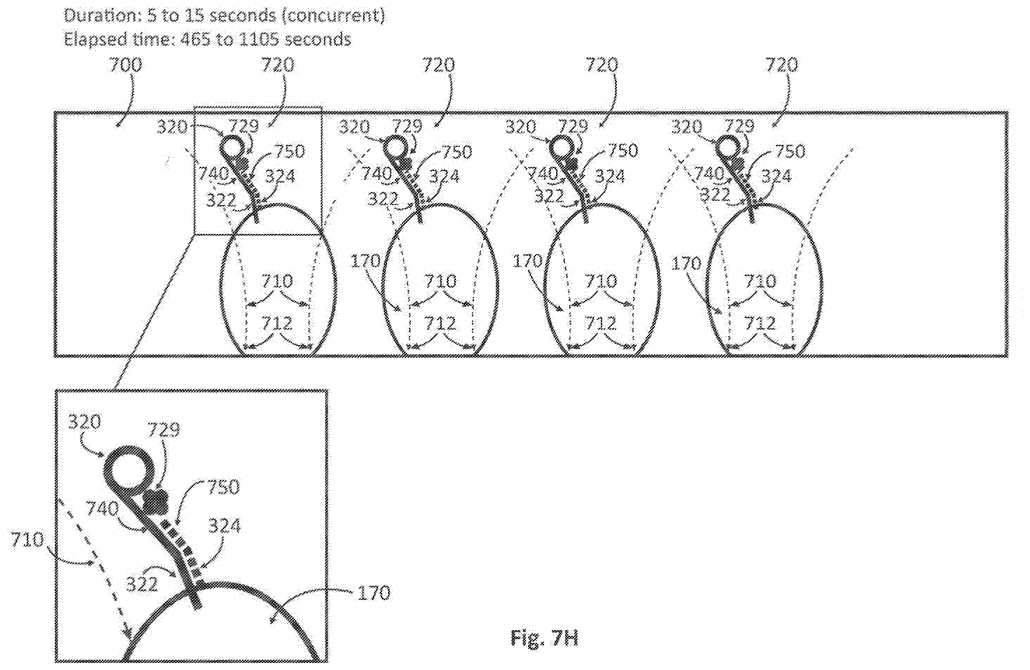

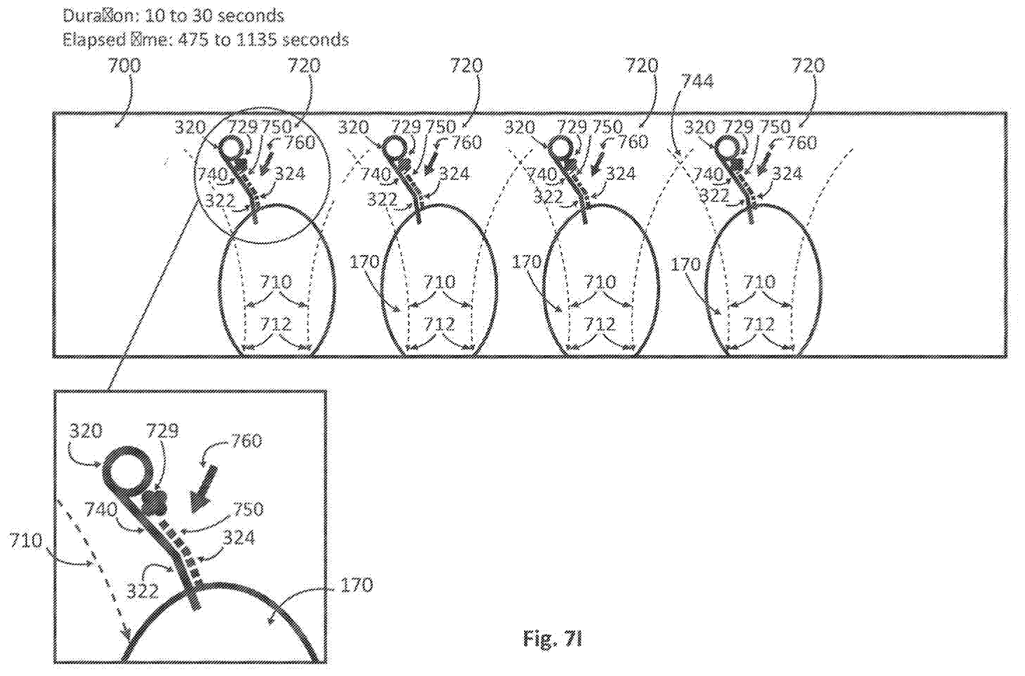

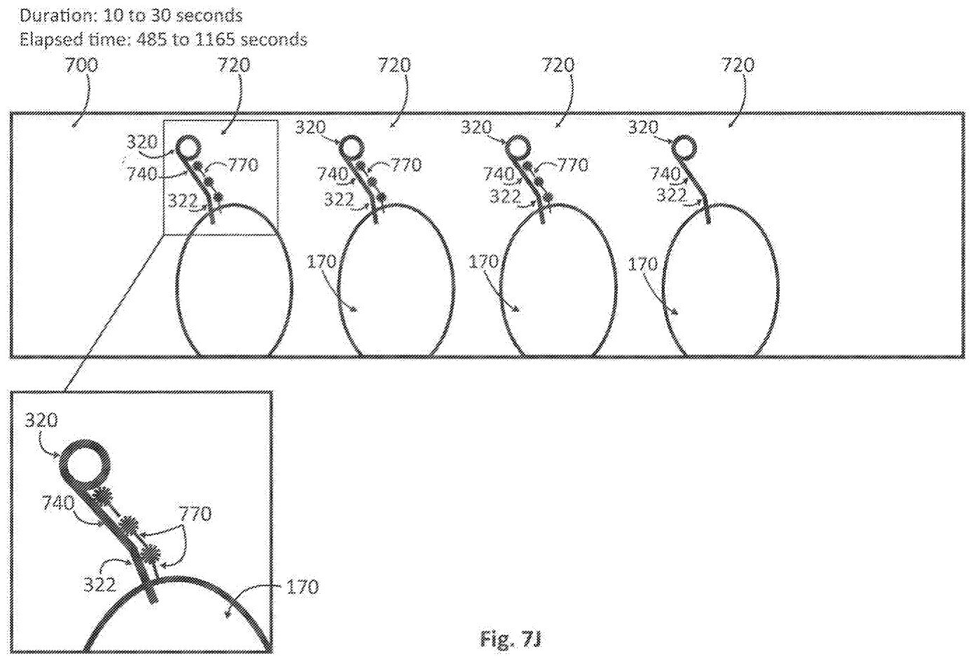

Reference is now made to FIGS. 1A and 1B, which are simplified pictorial assembled and exploded view illustrations of an electrophoretic array assembly 100 constructed and operative in accordance with a preferred embodiment of the invention, which defines a multiplicity of immobilized, mutually spaced and mutually electrically separated target molecule-specific microgel regions during rolling-circle amplification (RCA) operation, and to FIGS. 2A and 2B, which are simplified pictorial assembled and exploded view illustrations of the electrophoretic array assembly of FIGS. 1A and 1B in which the multiplicity of immobilized, mutually spaced anti mutually electrically separated microgel regions are shown in a dehydrated storage operative orientation. It is appreciated that electrophoretic array assembly 100 is particularly suitable for use in a method described hereinbelow with reference to FIGS. 7A-7I.

As seen in FIGS. 1A and 1B, the electrophoretic array assembly 100 comprises an enclosure defined by a substrate 110, a peripheral wall structure 120 and a window 130. Access to the interior of the enclosure is preferably provided by a solution ingress aperture 140 and a solution egress aperture 150 formed in substrate 110.

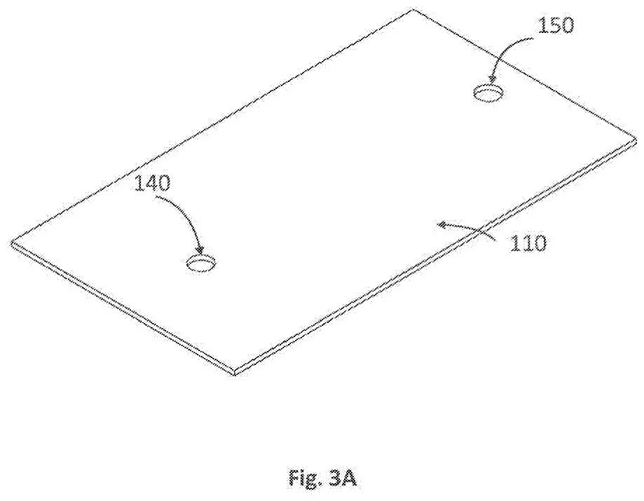

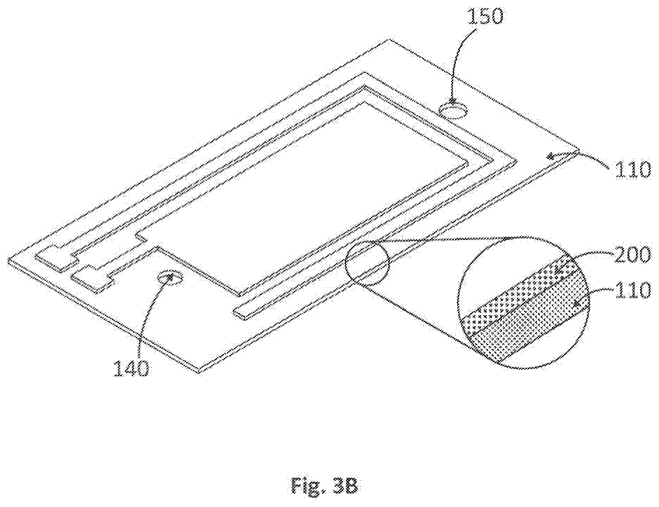

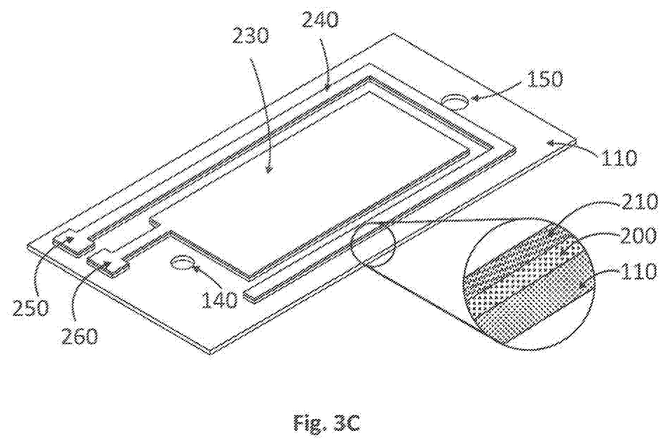

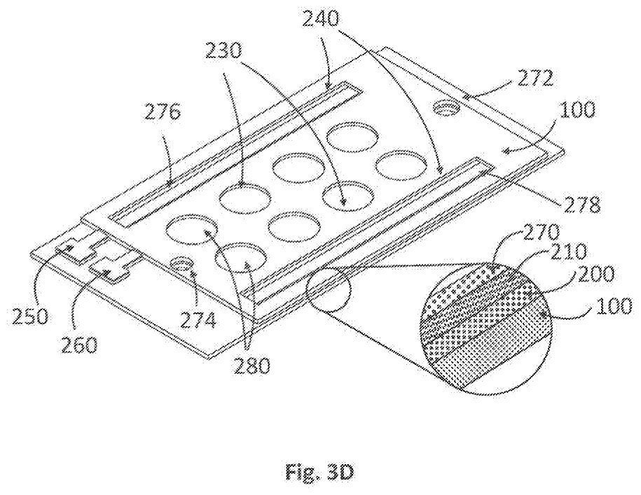

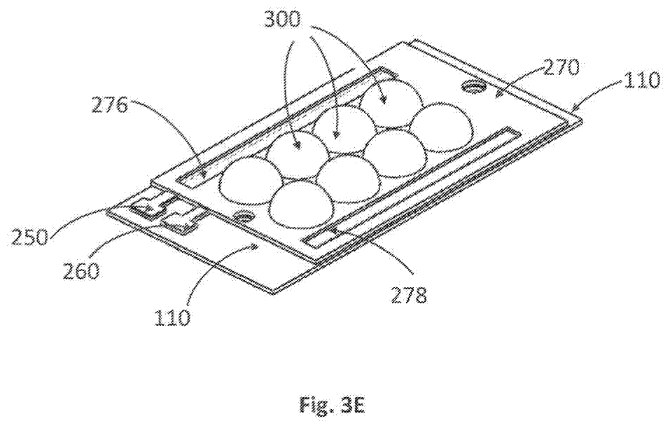

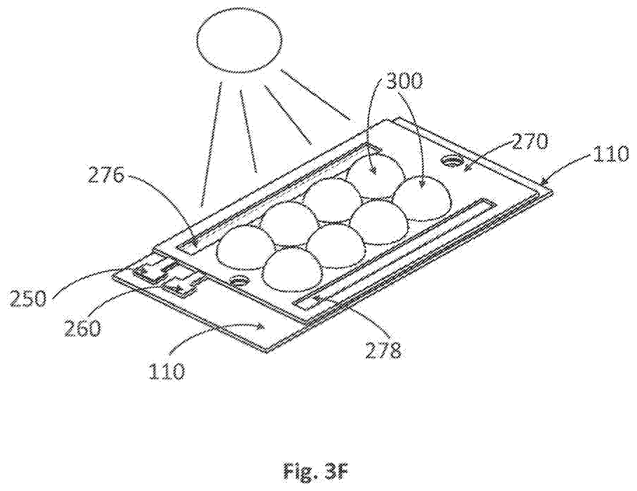

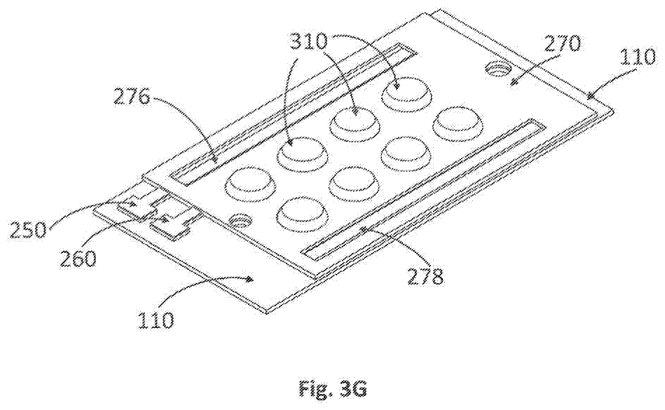

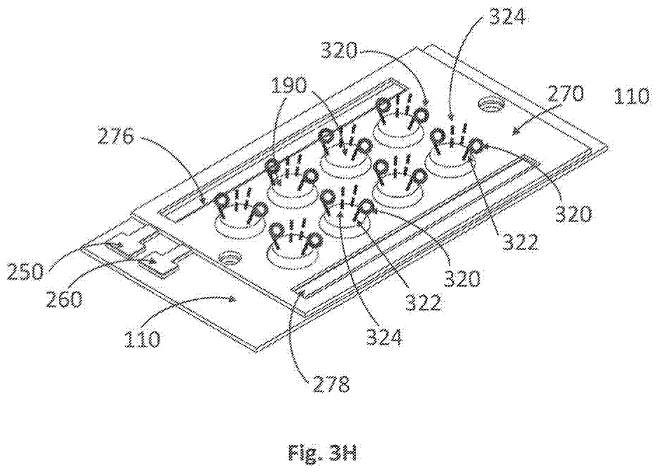

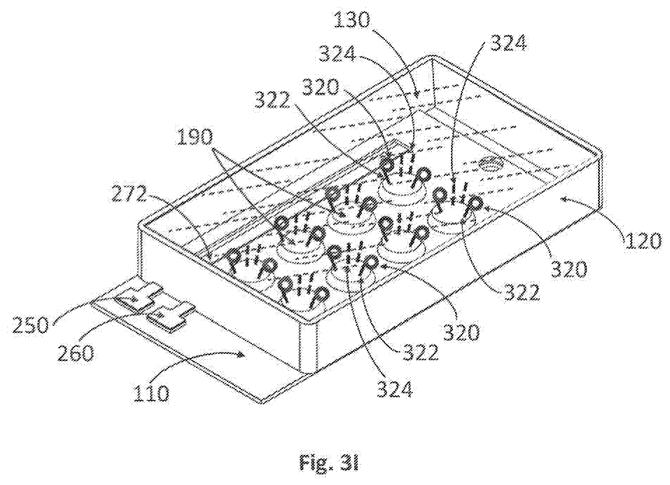

An electrophoretic array 160 is formed onto substrate 110, as will be described hereinbelow in greater detail with reference to FIGS. 3A-3I. A multiplicity of target molecule-specific microgel deposits 170, preferably having bound thereto different RCA circular probes, forward primers and reverse primers, here indicated generally by reference numeral 175 in a symbolic manner, are immobilized onto discrete working electrode locations 180, defined by the electrophoretic array 160. In FIGS. 1A and 1B. the target molecule-specific microgel deposits 170 are shown in an operative orientation suitable for rolling circle amplification.

In FIGS. 2A and 2B, the target molecule-specific microgel deposits are shown in a dehydrated state suitable for storage and are designated by reference numerals 190. If is appreciated that the target molecule-specific microgel deposits 190 of FIGS. 2A and 2B, when hydrated, by supply of a suitable solution, preferably a nucleic acid target molecule containing solution, to the interior of the electrophoretic array assembly 100, preferably assume the operative orientation shown in FIGS. 1A & 1B, where they are designated by reference numerals 170.

Preferred dimensions of the electrophoretic array assembly 100 and various components thereof, assuming, for ease of calculation, that each microgel deposit 170 exposed to solution assumes a generally hemispherical shape, are as follows:

Solution volume: approximately 100 mm³

Interior height between substrate 110 and window 130: 0.8 mm-2.0 mm

Height of target molecule-specific microgel deposits 170 above substrate 110 in the operative orientation of FIGS. 1A & 1B: 0.5 mm-1.25 mm

Height of target molecule-specific microgel deposits 190 above substrate 110 in the operative orientation of FIGS. 2A & 2B: 0.1 mm-0.3 mm

Surface area of each target molecule-specific microgel deposit 170 above substrate 110 in the operative orientation of FIGS. 1A and 1B: 0.0016-0.009 mm²

Ratio of surface area of each target molecule-specific microgel deposit 170 exposed to solution to solution volume: 0.000016-0.00009 mm² of exposed surface area of microgel deposit per mm³ of solution.

It is appreciated that the actual surface area and the actual ratio of surface area to solution volume are greater than or equal to the surface area and ratio calculated using the simplifying assumption of a hemispherical shape.

The structure and construction of the electrophoretic array assembly 100 will now be described with additional reference to FIGS. 3A-3I.

Turning initially to FIG. 3A, it is seen that substrate 110, is typically a polyester sheet, such as polyethylene terephlhalate, preferably of thickness 0.1 mm. Substrate 110 may be provided with solution ingress aperture 140 and solution egress aperture 150 at this stage, preferably by laser cutting. Alternatively, solution ingress aperture 140 and solution egress aperture 150 may be formed in substrate 110 at a later stage.

Turning now to FIG. 3B. it is seen that substrate 110 is formed with an initial patterned layer 200 of a highly conductive material, preferably by screen printing of Henkel 479SS ink. The thickness of layer 200 is preferably 0.03 mm. Layer 200 provides uniform electrical current conduction to each of the microgel deposits in the electrophoretic array assembly 100.

FIG. 3C shows subsequent forming of a carbon layer 210 in registration with the initial patterned layer 200, preferably by screen printing of DuPont 7102 and BQ 221. The thickness of layer 210 is preferably 0.03 mm. Layer 210 serves as the working electrode material, which is exposed to the solution as will be described hereinbelow. Layer 210 also controls the voltage applied between an inner working electrode 230 and an outer counter electrode 240.

Mutually registered layers 200 and 210 together define outer counter electrode 240 and inner working electrode 230, which are connected to respective electrical contacts 250 and 260.

Referring now additionally to FIG. 3D, it is seen that a patterned dielectric layer 270 is formed over layers 200 and 210 and over substrate 100, preferably by screen printing of CMI-101-80 79SS ink, commercially available from Creative Materials. Inc. (Ayer, Mass.). Dielectric layer 270 preferably has a thickness of 0.04 mm.

As seen in FIG. 3D, dielectric layer 270 is preferably formed with apertures 272 and 274, which preferably correspond in size and location to solution ingress aperture 140 and solution egress aperture 150. Dielectric layer 270 is also preferably provided with elongate apertures 276 and 278, which overlie parts of counter electrode 240. The length of each of elongate apertures 276 and 278 are preferably 100 mm. Additionally, dielectric layer 270 is formed with a multiplicity of apertures 280, here shown as an array of eight apertures 280, which overlie working electrode 230 and define therewith discrete working electrode locations 180 (FIGS. 1B and 2B). Preferably, apertures 280 are all identical circular apertures having a diameter of 0.2 mm-0.5 mm and a minimum spacing therebetween of 1 mm.

Turning now to FIG. 3E, it is seen that an array of microgel deposits 300 is formed, as by robotic spotting over working electrode locations 180. Microgel deposits 300 preferably have a generally hemispherical shape when hydrated and a diameter in the plane of the dielectric layer 270 of 0.70 mm so that they are mutually physically separated from each other. The microgel deposits 300 preferably have a height of between 0.5 mm and 1.2 mm and an exposed surface area of 0.0016-0.009 square millimeters. The microgel deposits 300 are preferably formed of (bis)acrylamide hydrogel containing streptavidin.

Turning now to FIG. 3F, it is seen that the microgel deposits 300 are preferably illuminated by UV illumination to polymerize components of the microgel deposits 300. Histidine is added to the microgel deposits 300 and the microgel deposits 300 are then washed to remove excess histidine.

Following polymerization, the microgel deposits 300 are dried as by air drying, producing dried microgel deposits 310, as seen in FIG. 3G.

Turning to FIG. 3H, it is seen that different nucleic acid target molecule specific RCA circular probes 320, and, preferably, also forward primers 322 and reverse primers 324 are bound to corresponding different ones of the dried microgel deposits 310, as by robotic spotting, thereby producing different target molecule-specific microgel deposits 100 (FIGS. 2A & 2B). It is appreciated that throughout the drawings, nucleic acid target molecule specific RCA circular probes 320, forward primers 322 and reverse primers 324 are shown symbolically and not to scale.

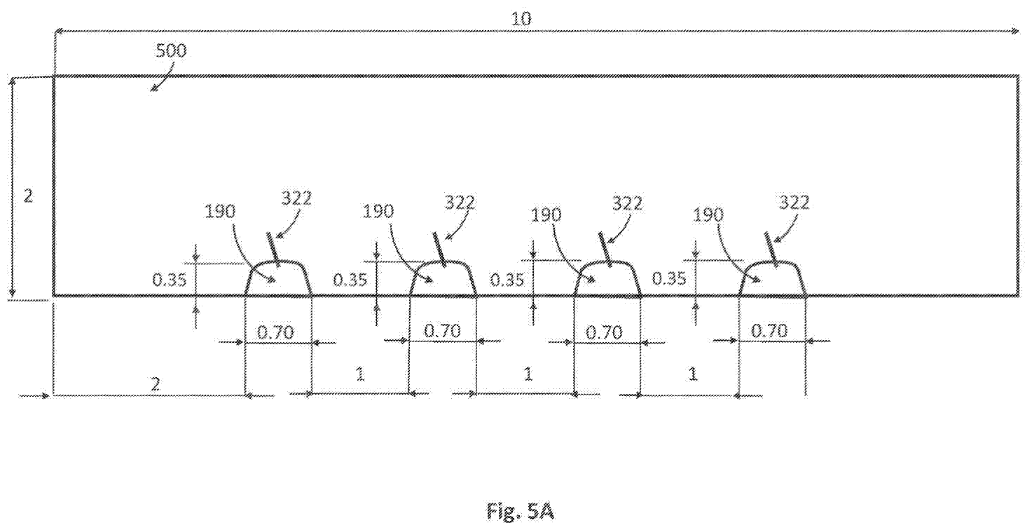

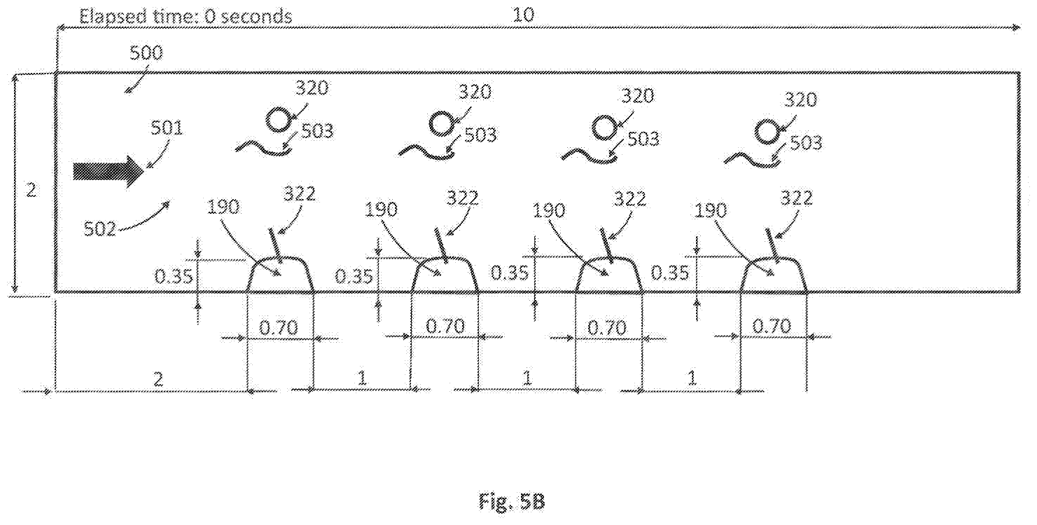

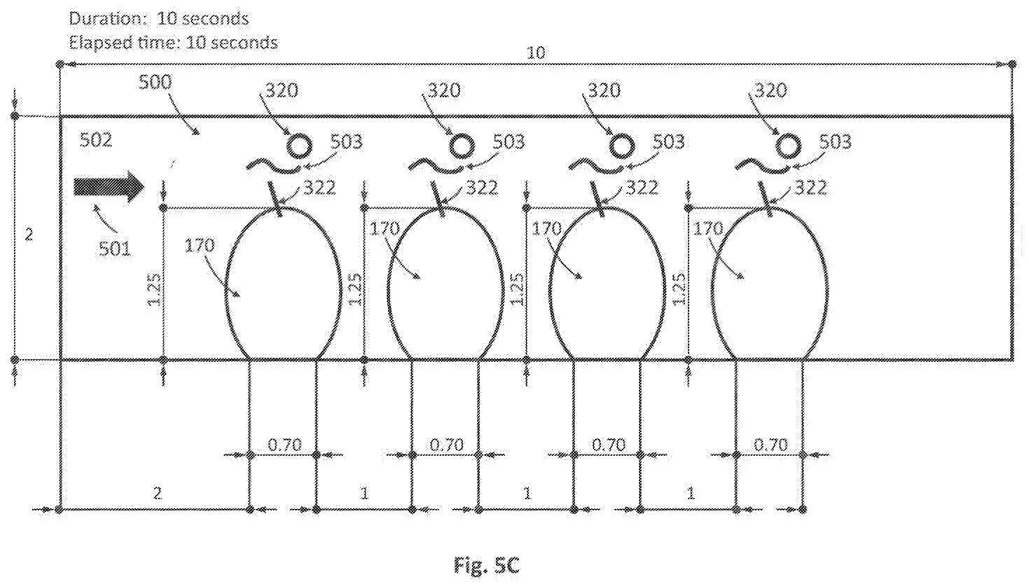

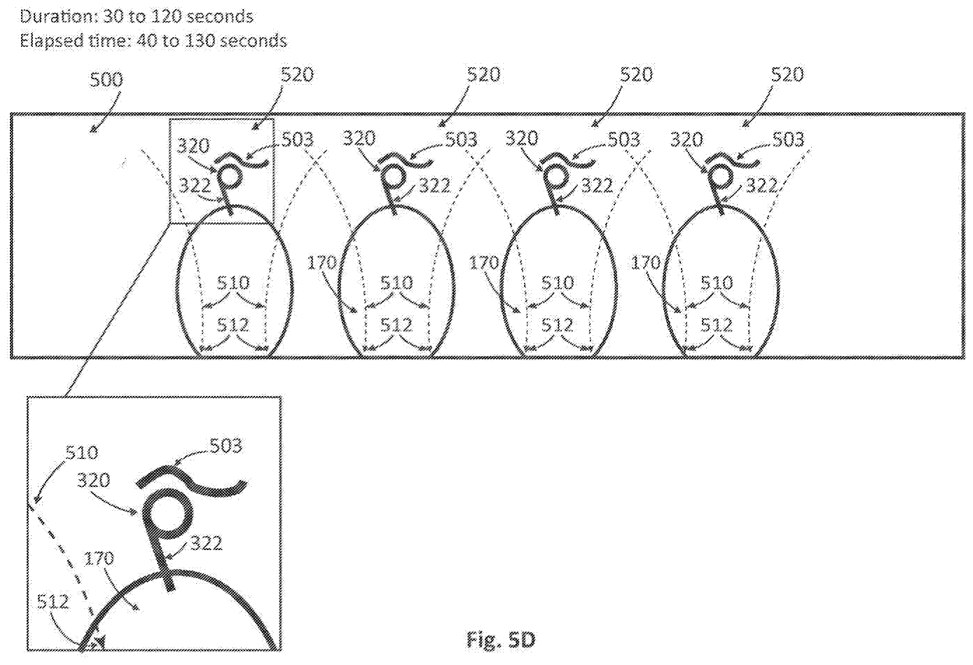

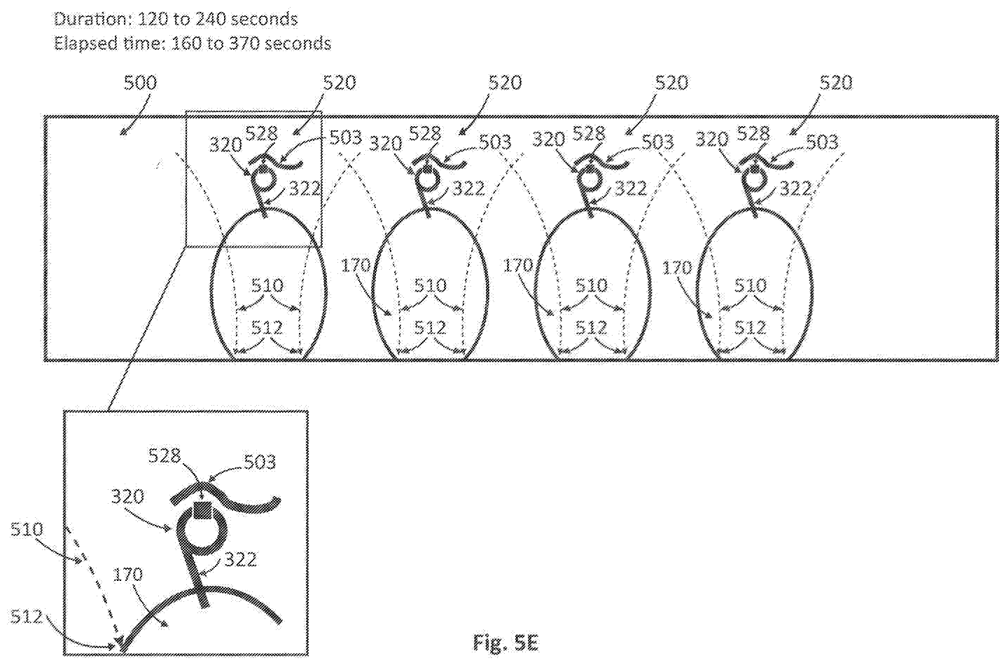

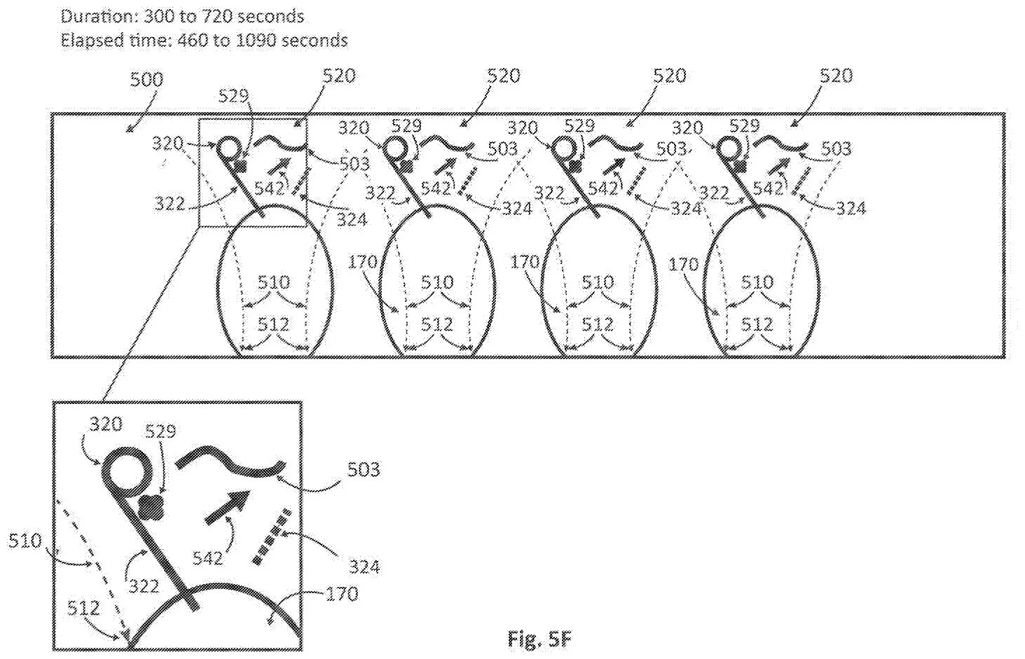

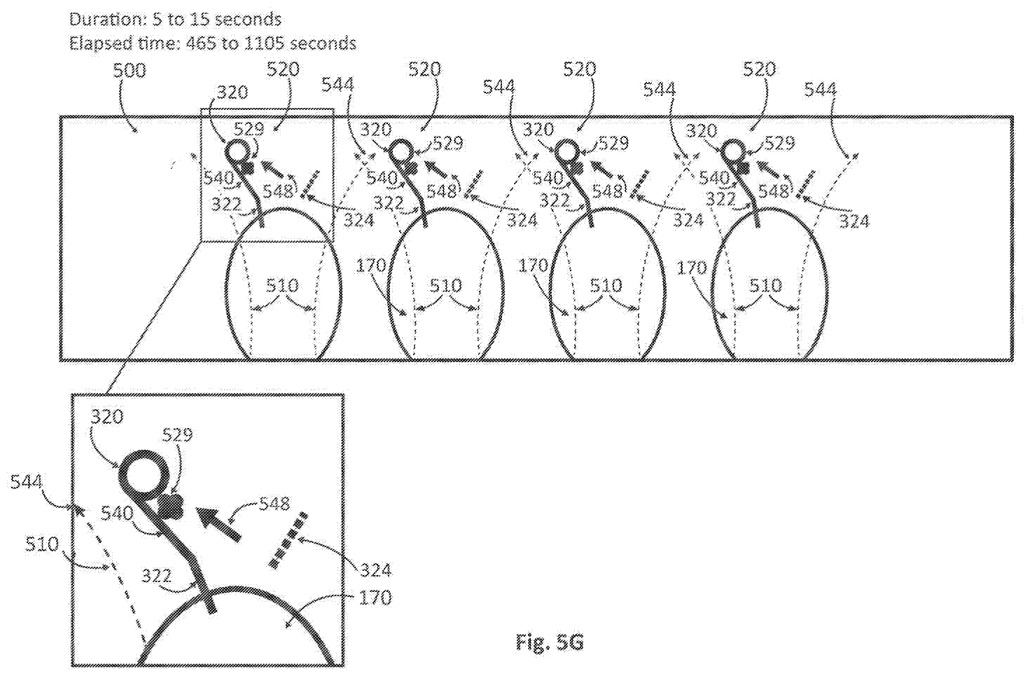

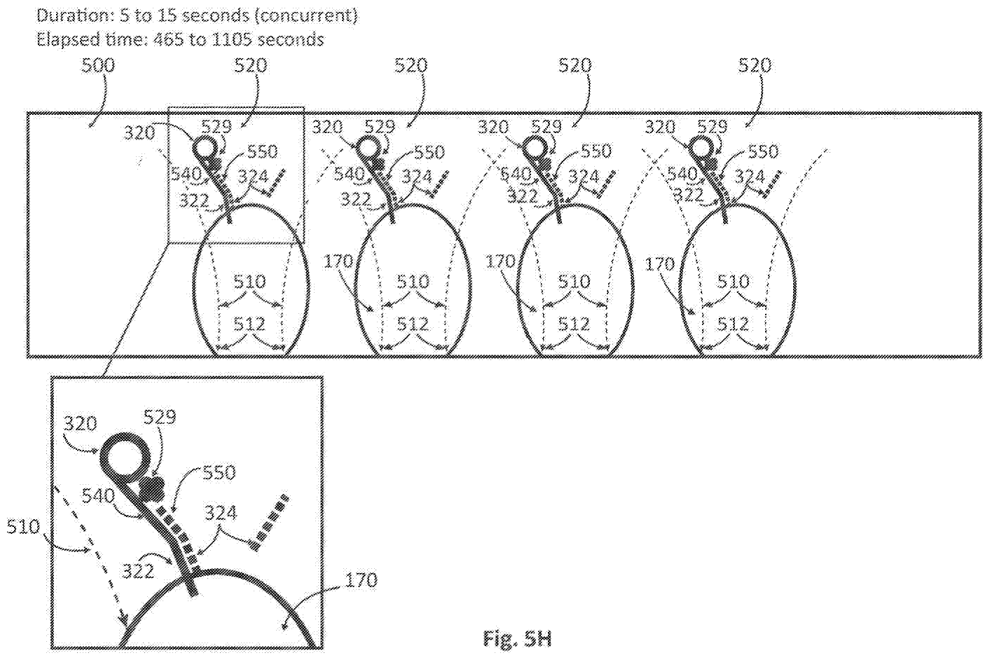

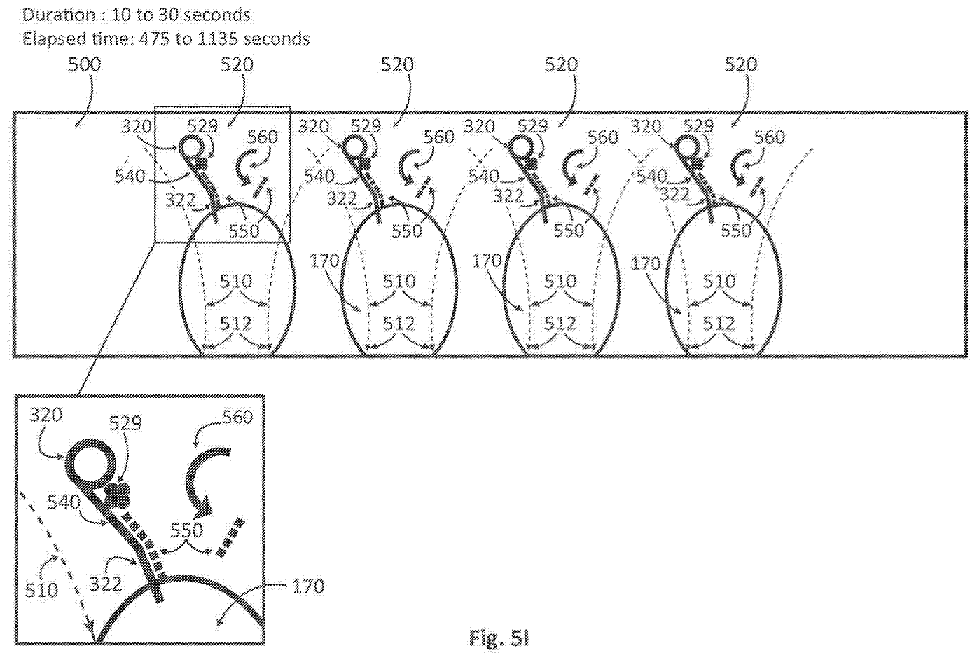

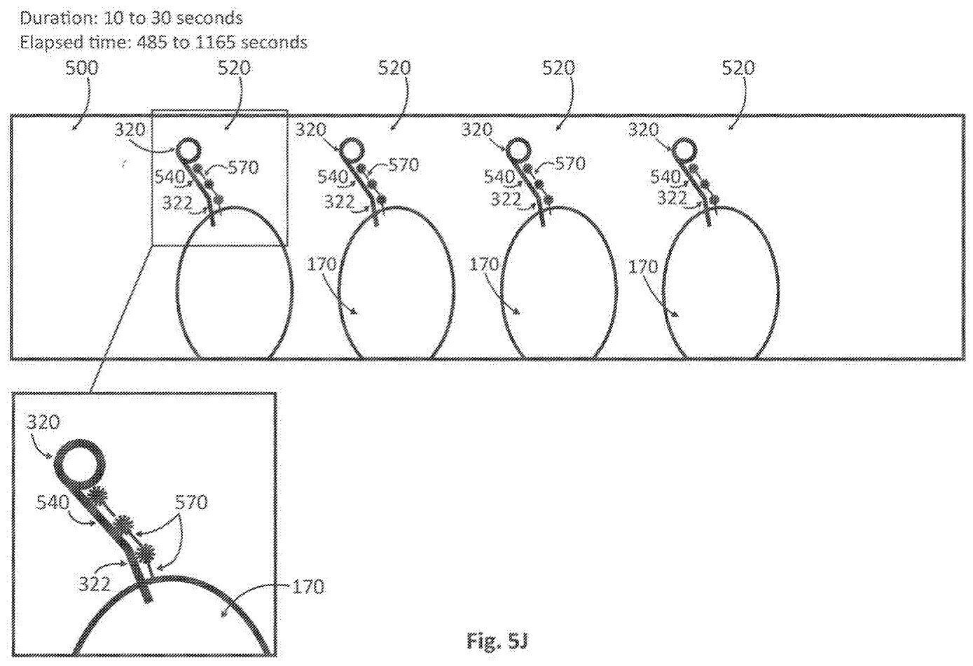

It is appreciated that although, in the embodiment shown in FIGS. 3H-3I and in FIGS. 1A-2B, nucleic acid target molecule specific RCA circular probes 320, forward primers 322 and reverse primers 324 are all shown as being bound to microgel deposits 310, in alternative embodiments one or more of nucleic acid target molecule specific RCA circular probes 320, forward primers 322 and reverse primers 324 may not be bound to microgel deposits 310 and may be provided in a solution together with the nucleic acid target molecules. In the embodiment shown in FIGS. 3H-3I and in the alternative embodiments, the nucleic acid target molecule specific RCA circular probes 320, forward primers 322 and reverse primers 324 are located in the immobilized, mutually spaced and mutually electrically separated target molecule-specific microgel regions, as described hereinbelow with reference to FIGS. 5A-7J.

As seen in FIG. 3I, following suitable washing of the target molecule-specific microgel dried microgel deposits 190 and the addition of raffinose as a preservative the peripheral wall structure 120 and the window 130 are assembled onto the substrate 110, thereby completing manufacture of the electrophoretic array assembly 100 in a shelf storable state, as described above with reference to FIGS. 2A & 2B. The electrophoretic array assembly 100 is ready for use in accordance with a preferred embodiment of die invention, which provides a method for rapid detection of the presence of at least one nucleic acid target molecule, from among a multiplicity of pre-selected nucleic acid target molecules, in a solution, including the steps of:

introducing the solution to at least a multiplicity of immobilized, mutually spaced and mutually electrically separated microgel regions on an electrophoretic array, each of the multiplicity of immobilized, mutually spaced and mutually electrically separated microgel regions containing a microgel deposit containing materials suitable for binding of a different one of the multiplicity of pre-selected nucleic acid target molecules and performing rolling circle amplification;

performing rolling circle amplification at least generally simultaneously at each of the immobilized, mutually spaced and mutually electrically separated microgel regions, while applying electric fields thereto during various stages of the rolling circle amplification; and

detecting the presence of at least one of the multiplicity of pre-selected nucleic acid target molecules at at least one corresponding one of the immobilized, mutually spaced and mutually electrically separated microgel regions.

wherein the detecting occurs within a short time period of the introducing, the short time period preferably being less than 30 minutes, more preferably less than 25 minutes and even more preferably less than 20 minutes.

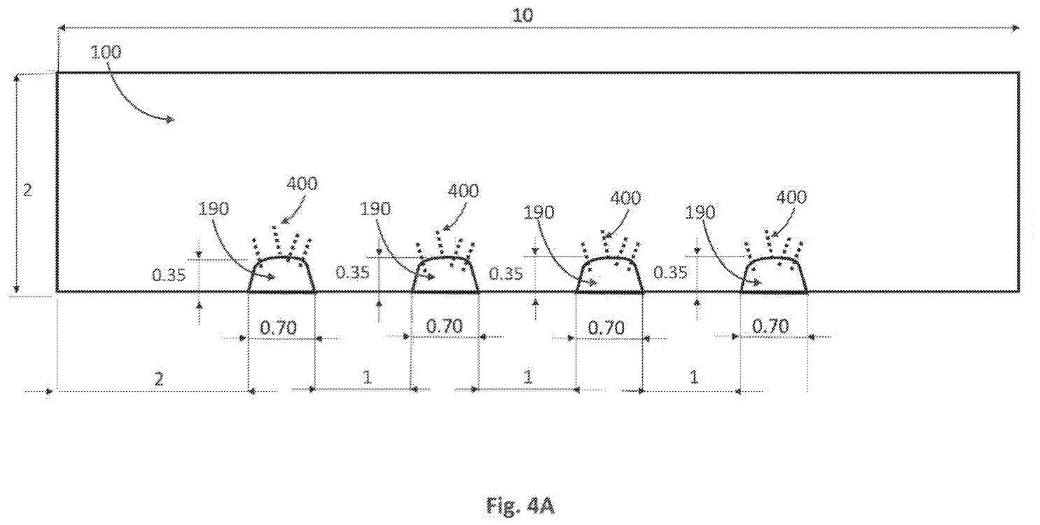

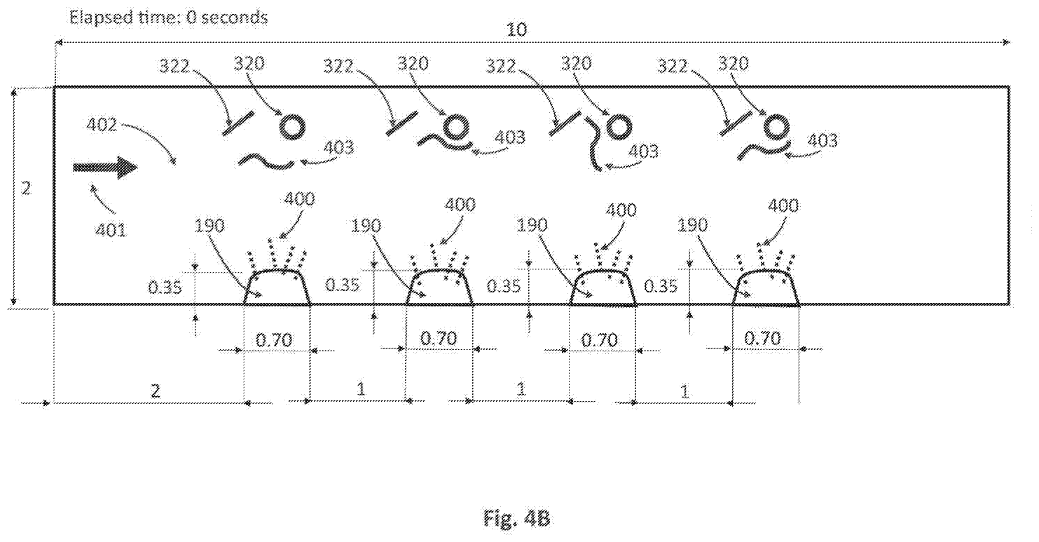

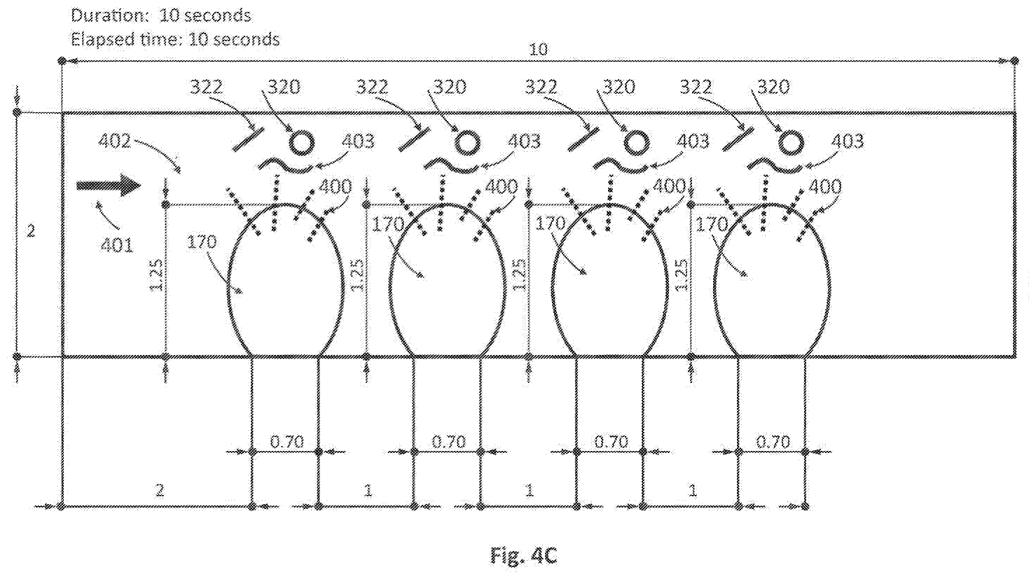

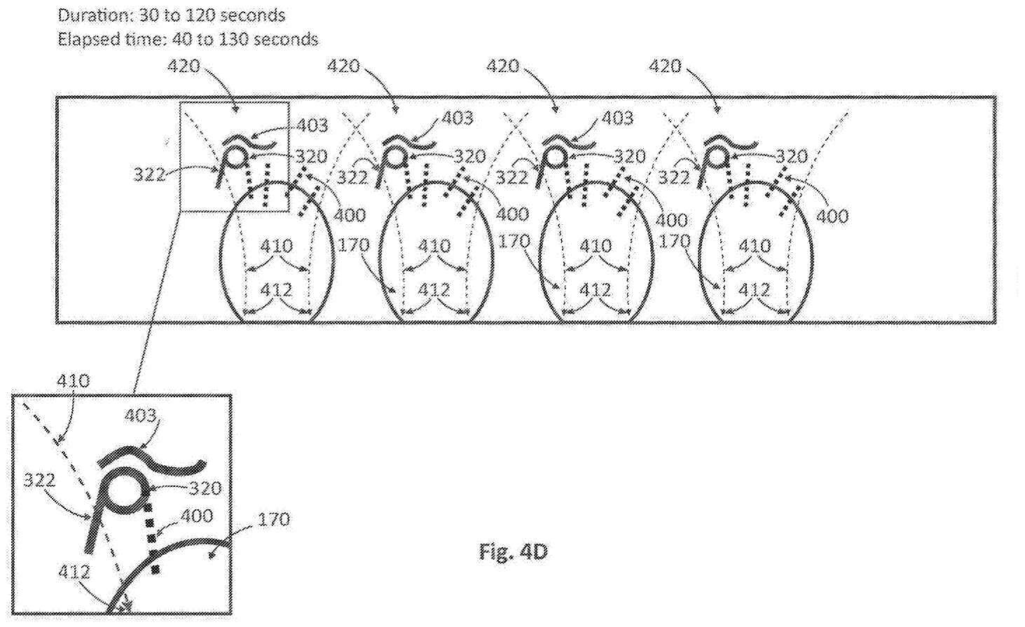

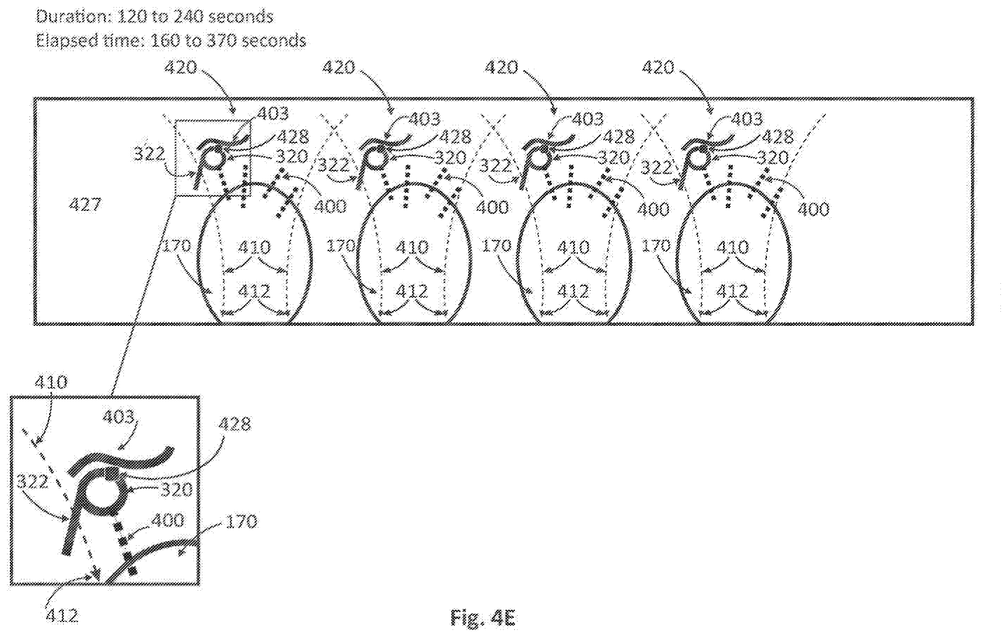

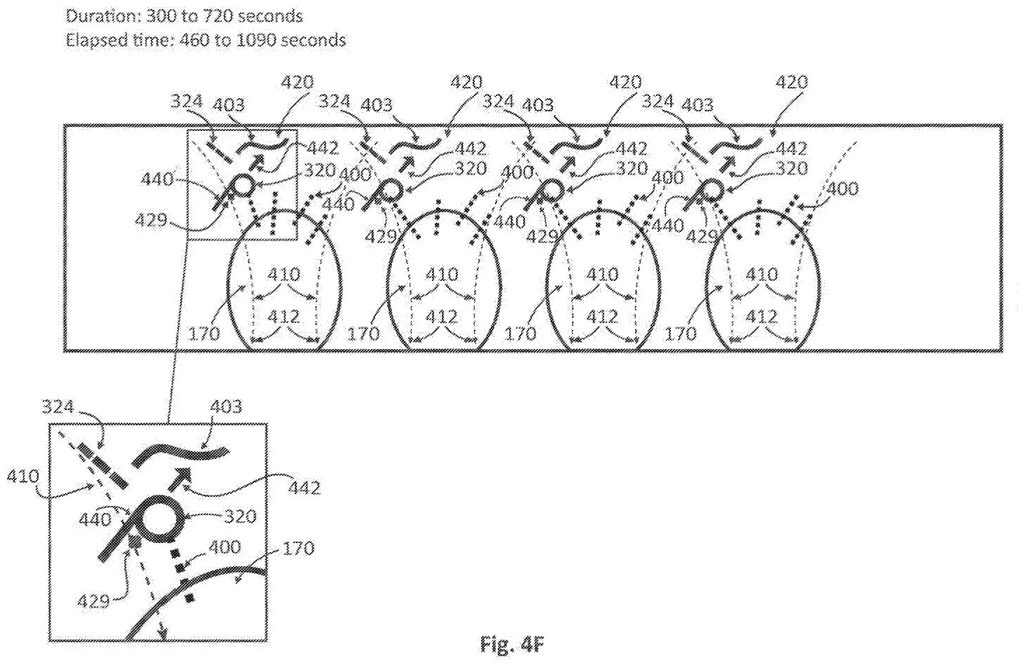

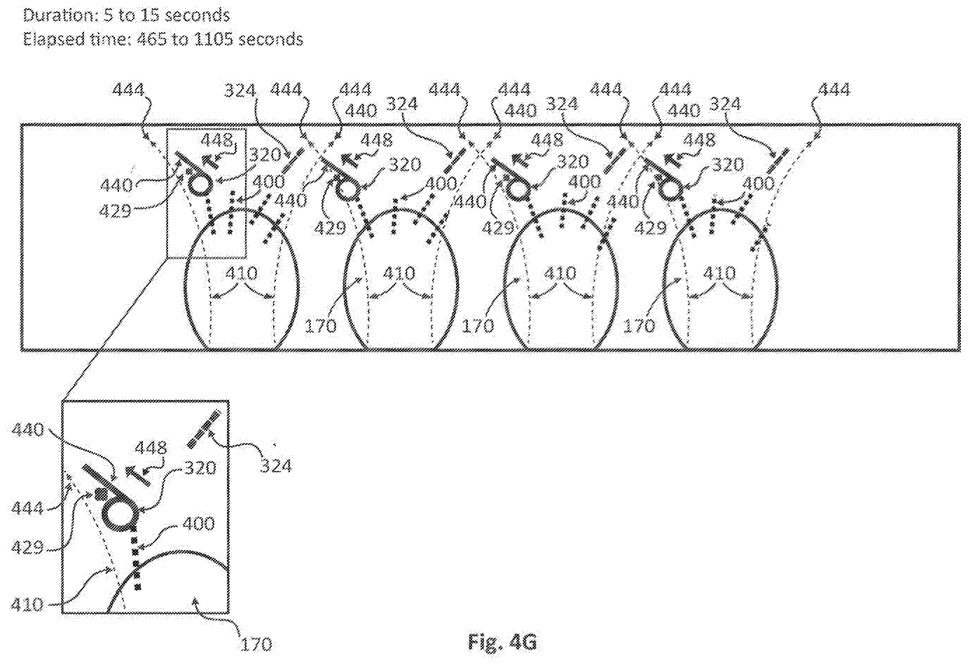

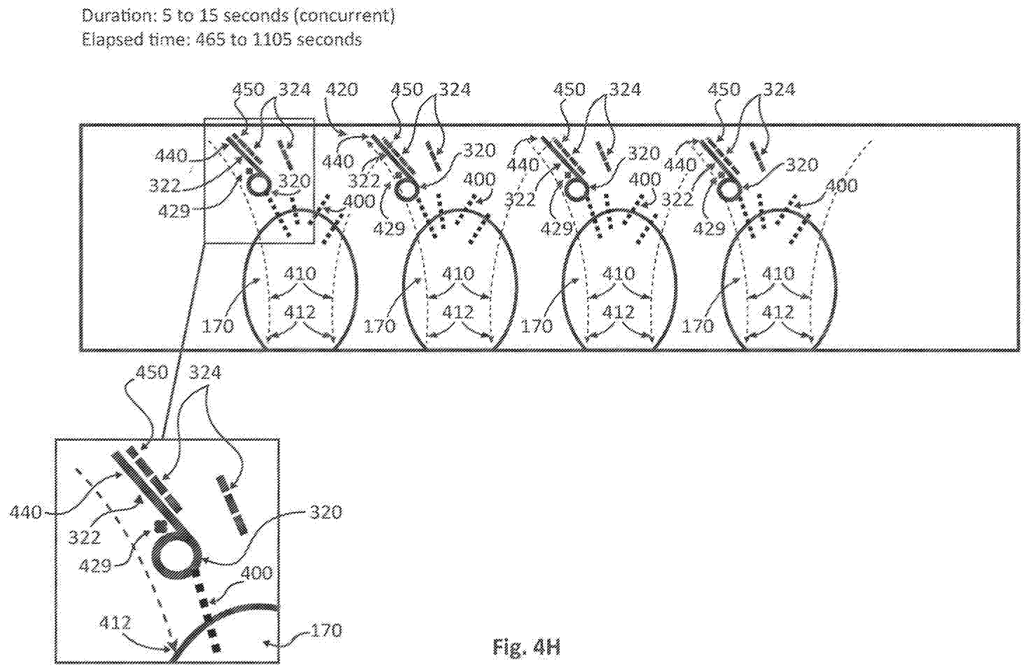

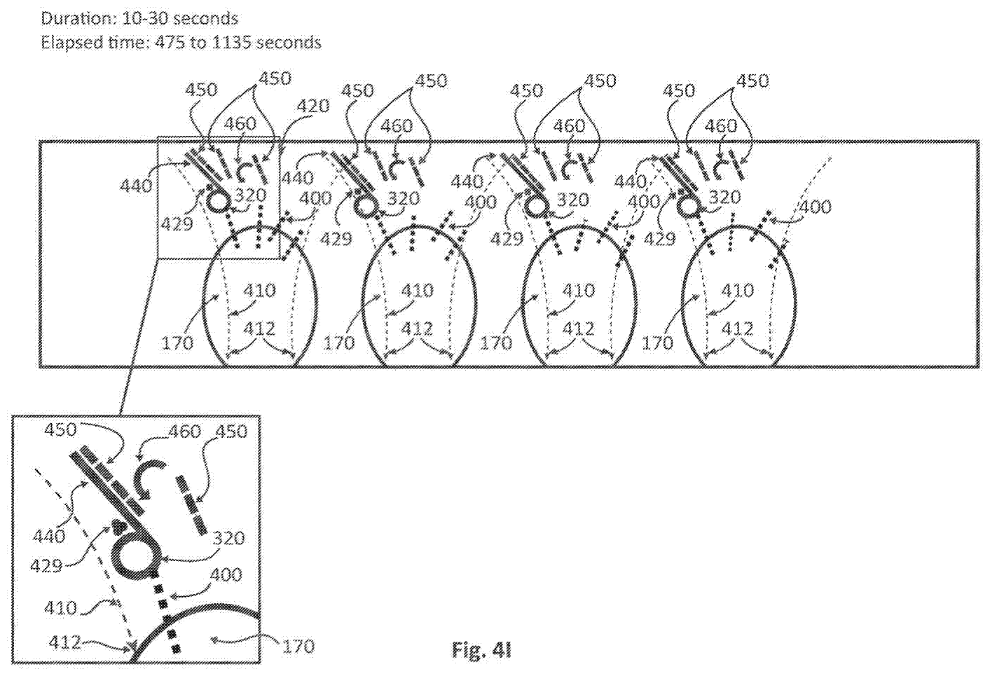

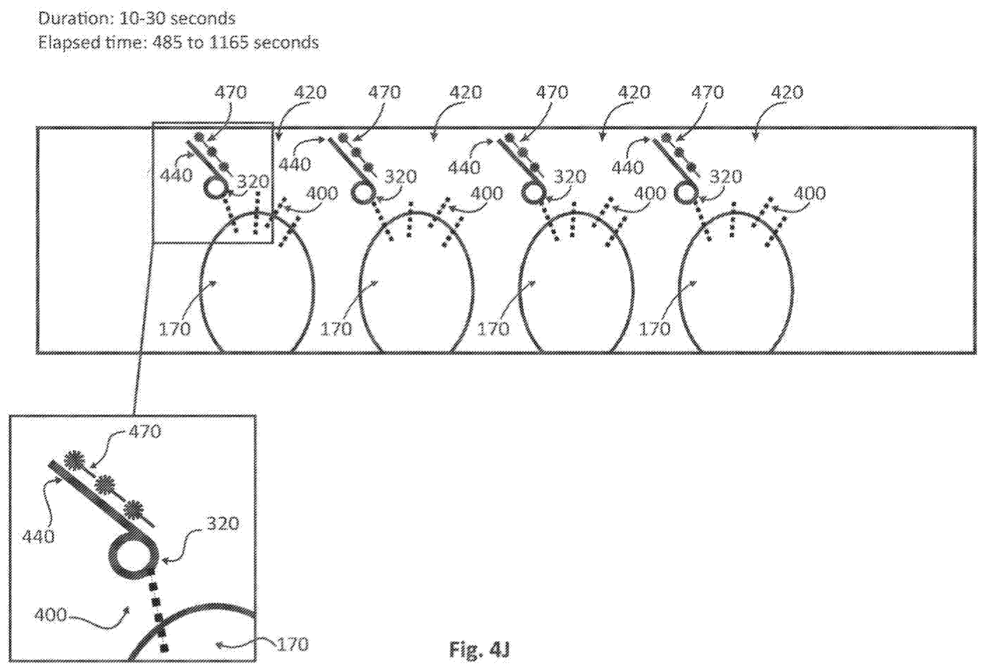

In the description which follows, four variations of carrying out the above method are described in detail with reference to FIGS. 4A-4J, FIGS. 5A-5J, FIGS. 6A-6J and FIGS. 7A-7J, respectively. The electrophoretic array assembly 100 described hereinabove is particularly suitable for use in carrying out the method of FIGS. 7A-7J.

All of these methods employ rolling circle amplification. Rolling circle amplification is a known technique and is described, inter alia, in the following publications, the disclosures of which are hereby incorporated by reference:

U.S. Pat. No. 5,854,033; Lizardi el al., Nature Genetics 19(3):225-232 (1998),

Michael G. Mohsen and Eric T. Kool, The Discovery of Rolling Circle Amplification and Rolling Circle Transcription. Acc Chem Res. 2016, 49(11): 2540-2550

Peiying Feng, el al., Identification and Typing of Isolates of Cyphellophora and Relatives by Use of Amplified Fragment length Polymorphism and Rolling Circle Amplification. Journal of Clinical Microbiology, 2013 Volume 51 Number 3. p. 931-937.

Signal Amplification by Rolling Circle Amplification on DNA Microarrays. G. Nallur et al., Nucleic Acids Research. 2001, Vol. 29, Vol. 29, No. 123, e118

M. Monsur Ali et al., Rolling circle amplification: a versatile tool for chemical biology, materials science and medicine. Chemical Society Review, Chem. Soc. Rev. 2014, 43, 3324-3341.

Ali M M. Li F, Zhang Z, Zhang K. Kang D. Ankrum J A, Le X C, Zhao W. Rolling circle amplification: a versatile tool for chemical biology, materials science and medicine. Chem Soc Rev. 2014; 43:3324.

Kool, E T. Rolling circle synthesis of oligonucleotides and amplification of select randomized circular oligonucleotides. U.S. Pat. No. 5,714,320. Feb 3. 1998.

Fire A. Xu S. Rolling replication of short DNA circles. Proc Natl Acad Sci USA. 1995; 92:4641-4645.

Nilsson M. Malmgren H. Samiotaki M. Kwiatkowski M. Chowdhary B P, Landegren U. Padlock Probes: Circularising Oligonucleotides for Localized DNA Detection. Science. 1994; 265:2085-2088.

The various methods which are described hereinbelow include features which are novel and unobvious in view of the prior art rolling circle amplification techniques.

Reference is now made to FIGS. 4A-4J, which illustrate principal stages in rapid detection of the presence of at least one nucleic acid target molecule, from among a multiplicity of pre-selected nucleic acid target molecules in accordance with one preferred embodiment of the present invention.

The method of FIGS. 4A-4J is preferably carried out using electrophoretic array assembly 100 in a shelf storable state, as described above with reference to FIGS. 2A & 2B, wherein only capture probes are bound to the immobilized, mutually spaced and mutually electrically separated microgel deposits 190. It is appreciated that for simplicity, the substrate 110, the peripheral wall structure 120 and the window 130 as well as the various layers constituting the electrophoretic array 160, as described hereinabove with reference to FIGS. 1A-2B, are not specifically shown in FIGS. 4A-4J. Each of FIGS. 4A-4J is a simplified side view sectional illustration taken generally along lines 4-4 in FIG. 2A.

FIG. 4A shows the electrophoretic array assembly 100 in its shelf storable state as shown in FIG. 2A, with symbolic, not to scale, indications of various different oligonucleotide nucleic acid specific capture probes 400, such as those specific but not limited to identification of meningitis infectious disease pathogens, or other diseases, which are bound to corresponding different ones of the dried microgel deposits 190. FIG. 4A indicates, in millimeter units, the interior dimensions of a preferred embodiment of the electrophoretic array assembly 100, as well as the general dimensions of the immobilized dried target molecule-specific microgel deposits 190, which are centered on but extend somewhat beyond working electrode locations 180 (FIGS. 1A-3I).

FIG. 4B illustrates the introduction, symbolized by an arrow 401, of a solution 402 containing one or more different types of nucleic acid target molecules 403, so as to fill the interior of the electrophoretic array assembly 100. Solution 402 preferably includes, in addition to nucleic acid target molecules 403, forward primers 322 and nucleic acid target molecule specific RCA circular probes 320.

Preparation of solution 402 is not part of the present claimed invention and is earned out in accordance with conventional techniques, such as those described in “Nasir Ali, Rita de Cássia Pontello Rampazzo, Alexandre Dias Tavares Costa, and Marco Aurclio Krieger, Current Nucleic Acid Extraction Methods and Their Implications to Point-of-Care Diagnostics. BioMed Research International Volume 2017, Article ID 9306564, 13 pages”. Solution 402 preferably includes a low conductivity eluent liquid, typically introduced during preparation of the solution 402, that promotes electronic addressing of nucleic acids and promotes activity of restriction enzymes in solution 402. A preferred eluent liquid includes histidine and a restriction enzyme buffer. FIG. 4B shows the dried target molecule-specific microgel deposits 190 in their dehydrated state. The introduction of solution 402 into electrophoretic array assembly 100, so that solution 402 contacts dried target molecule-specific microgel deposits 190, defines a time T0 and causes dried target molecule-specific microgel deposits 190 to assume their hydrated slate, designated by reference numeral 170 (FIGS. 1A & 1B).

FIG. 4C illustrates immobilized larger molecule-specific microgel deposits 170 in a hydrated state, as the result of the introduction of solution 402, containing nucleic acid target molecules 403, which fills the interior of the electrophoretic array assembly 100. FIG. 4C illustrates that the hydrated target molecule-specific electrically separated microgel deposits 170 have a typical maximum height of 1.25 mm. The hydration of the target molecule-specific microgel deposits 170 to their state shown in FIG. 4C preferably takes about 10 seconds and is preferably completed at time T=T0+10 seconds.

FIG. 4D illustrates electrophoretic addressing of nucleic acids to the hydrated immobilized, mutually electrically separated microgel deposits 170 in the presence of a DC electric field preferably of 10-300 Volts per centimeter applied between working electrode locations 180 on working electrode 230 and counter electrode 240 (FIGS. 1A & 1B). Portions of lire electric field lines are indicated by reference numerals 410 and the direction of the electric field is indicated by arrows 412. It may be appreciated that the electric field lines define three-dimensional, immobilized, mutually spaced and mutually electrically separated target molecule-specific microgel regions 420, each centered about a different target molecule-specific microgel deposit 170.

It is appreciated that addressing, as well as the various steps described hereinbelow with reference to FIGS. 4E-4J, occur not only on the surface of microgel deposits 170, but also within the volume of microgel deposits 170.

As seen in FIG. 4D, the application of the DC electric field to the three-dimensional, immobilized, mutually spaced and mutually electrically separated target molecule-specific microgel regions 420 causes rapid transport of nucleic acid target molecules 403, nucleic acid target molecule specific RCA circular probes 320 and forward primers 322 to the target molecule-specific microgel deposits 170, which in tum facilitates specific hybridization between the nucleic acid target molecules 403 and the nucleic acid target molecule specific RCA circular probes 320, specific hybridization between the forward primers 322 and the nucleic acid target molecule specific RCA circular probes 320 and capture of the nucleic acid target molecule specific RCA circular probes 320 by target molecule-specific capture probes 400, which capture probes 400 are bound to hydrated target molecule-specific electrically separated microgel deposits 170.

The duration of the stage illustrated in FIG. 4D is between 30 seconds and 120 seconds. The stage illustrated in FIG. 4D is preferably completed at a time T−T0+[40 to 130] seconds.

Reference is now made to FIG. 4K, which illustrates a ligation stage that normally follows the addressing stage shown in FIG. 4D and preferably takes place in the presence of a DC electric field, preferably of 10-300 Volts per centimeter in the same direction as the electric field in the step of FIG. 4D. The ligation stage preferably occurs in the presence of a ligation enzyme 428, for example. T-4 ligase, which is introduced into electrophoretic array assembly 100 through a solution. The duration of the ligation stage is typically 120 to 240 seconds. The ligation stage is preferably completed at T=T0+[160 to 370] seconds.

Reference Ls now made to FIG. 4F, which illustrates an RCA polymerization stage that normally follows the ligation stage shown in FIG. 4E and preferably occurs in the presence of a DC electric field, preferably of 10-300 Volts per centimeter in the same direction as the electric field in the step of FIG. 4E.

The RCA polymerization stage preferably occurs in the presence of a Bst polymerase enzyme 429, dNTPs (not shown) and a reverse primer 324, which are introduced into electrophoretic array assembly 100 through a solution, and forward primer 322, which is bound to RCA circular probe 320, which in turn bound to capture probe 400, which in turn is bound to target-molecule specific microigel deposit 170, preferably at a temperature of 65 degrees Celsius. A result of the RCA polymerization stage is generation of long RCA amplicons 440. As seen in FIG. 4F, following binding of polymerase enzyme 429 to nucleic acid target molecule specific RCA circular probes 320, nucleic acid target molecules 403 are displaced from nucleic acid target molecule specific RCA circular probes 320, as indicated by arrows 442. The duration of the RCA polymerization stage is typically 300 to 720 seconds. The RCA polymerization stage is preferably completed at T=T0+[460 to 1090] seconds.

Reference is now made to FIG. 4G, which illustrates an amplicon stretching stage that normally occurs during the RCA polymerization stage of FIG. 4F and preferably occurs in the presence of a DC electric field, preferably of 10-300 Volts per centimeter in a direction opposite to that of the electric field in the step of FIG. 4F as indicated by arrows 444. In the illustrated embodiment, stretching of amplicon 440 occurs in a direction indicated by an arrow 448. The amplicon stretching Mage preferably occurs in the presence of a Bst polymerase enzyme 429, dNTPs (not shown) and reverse printer 324 at a temperature of 65 degrees Celsius. The duration of the amplicon stretching stage is typically 5 to 15 seconds. The RCA polymerization stage is preferably completed at T=T0+[465 to 1105] seconds.

It is appreciated that the stages shown in FIGS. 4F and 4G may be repeated intermittently multiple times, with multiple stages shown in FIG. 4F each being of a shorter duration than that indicated above and being separated by a stage shown in FIG. 4G.

Reference is now made to FIG. 4H, which illustrates an exponential RCA amplification stage that normally occurs during the RCA polymerization stage of FIG. 4F and the amplicon stretching stage of FIG. 4G and preferably occurs in the presence of a DC electric field, preferably of 10-300 Volts per centimeter, which is normally in an opposite direction from that of the electric field in the step of FIG. 4G, as indicated by arrow 412, but preferably includes short periods in which the polarity of the electric field is reversed. In this stage, additional amplicon 450 are generated using reverse primers 324.

The exponential RCA amplification stage preferably occurs in the presence of a Bst polymerase enzyme 429, dNTPs (not shown) and reverse primer 324 at a temperature of 65 degrees Celsius. The duration of the exponential RCA amplification stage, which occurs during the RCA polymerization stage of FIG. 4F and the amplicon stretching stage of FIG. 4G, is typically 5-15 seconds. The RCA polymerization stage of FIG. 4F, the amplicon stretching stage of FIG. 4G and the exponential RCA amplification stage of FIG. 4H, preferably are completed at T=T0+[465 to 1105] seconds.

Reference is now made to FIG. 4I, which illustrates a post-RCA polymerization addressing stage that normally occurs following the completion of RCA polymerization stage of FIG. 4F, the amplicon stretching stage of FIG. 4G and the exponential RCA amplification stage of FIG. 4H, and preferably occurs in the presence of a DC electric field, preferably of 10-300 Volts per centimeter, which is normally in the same direction as that of the electric field in the step of FIG. 4H. The post-RCA polymerization addressing stage is particularly useful for concentrating amplicons 440 that are in solution 402 at a distance from target molecule-specific microgel deposits 170 and recapturing them at the target molecule-specific microgel deposits 170, as indicated by arrows 460, and preferably gathering the amplicons 440 at one location within each microgel region 420. The duration of the post-RCA polymerization addressing stage is typically 10-30 seconds. The post RCA polymerization addressing stage is preferably completed at T=T0+[475 to 1135] seconds.

Reference is now made to FIG. 4J, which illustrates a reporting stage that normally follows the post RCA polymerization addressing stage. The reporting stage preferably occurs in the presence of fluorescence reporters 470 complementary to amplicons 440 and 450, which is introduced to electrophoretic array assembly 100 through a solution. The duration of the reporting stage is typically 10-30 seconds. The reporting stage is preferably completed at T=T0+[485 to 1165] seconds.

Upon completion of the reporting stage and a subsequent washing stage, not shown, the detection of the presence of at least one nucleic acid target molecule, from among a multiplicity of pre-selected nucleic acid target molecules, may be carried out by conventional fluorescence detection techniques. It is thus appreciated that detection of at least one nucleic acid target molecule is preferably completed within between 8 minutes and 20 minutes of the initial supply of solution 402 to the interior of the electrophoretic array assembly 100.

It is appreciated that if preparation of solution 402 is completed within 4-5 minutes of acquisition of a sample, av by taking a blood sample from a patient, detection at least one nucleic acid target molecule may be completed within 12-25 minutes from sample acquisition.

Reference is now made to FIGS. 5A-5J, which illustrate principal stages in rapid detection of the presence of at least one nucleic acid target molecule, from among a multiplicity of pre-selected nucleic acid target molecules in accordance with another preferred embodiment of the present invention.

The method of FIGS. 5A-5J is preferably carried out using an electrophoretic array assembly 500 in a shelf storable state, as described above with reference 10 FIGS. 2A & 2B, wherein forward primers 322 are bound to the immobilized and mutually spaced microgel deposits 190. It is appreciated that for simplicity, the substrate 110, the peripheral wall structure 120 and the window 130 as well as the various layers constituting the electrophoretic array 160 are not specifically shown. Each of FIGS. 5A-5J is a simplified side view sectional illustration taken generally along lines 4-4 in FIG. 2A.

FIG. 5A shows an electrophoretic array assembly 500 in its dried, dehydrated, operative orientation, similar to that shown in FIG. 2A, with symbolic, not to scale, indications of various different oligonucleotide forward primers 322, which are bound to corresponding different ones of the dried microgel deposits 190.

FIG. 5A indicates, in millimeter units, the interior dimensions of a preferred embodiment of tire electrophoretic array assembly 500, as well as tire general dimensions of the immobilized dried target molecule-specific microgel deposits 190.

It is appreciated that the method of FIGS. 5A-5J differs from the method of FIGS. 4A-4J in that instead of capture probes 400 being bound to the immobilized dried target molecule specific microgel deposits 190 in the method of FIGS. 4A-4J. forward primers 322, which also serve as capture probes, are bound to the immobilized dried target molecule specific microgel deposits 190 in the method of FIGS. 5A-5J.

FIG. 5B illustrates the introduction, symbolized by an arrow 501, of a solution 502 containing nucleic acid target molecules 503, so as to fill the interior of the electrophoretic array assembly 500. This solution preferably includes nucleic acid target molecule specific RCA circular probes 320 in addition to nucleic acid target molecules 503.

Preparation of solution 502 is not pan of the present claimed invention and is carried out in accordance with conventional techniques, such as those described in “Nasir Ali, Rita de Cássia Pontello Rarapazzo, Alexandre Dias Tavares Costa, and Marco Aurelio Krieger. Current Nucleic Acid Extraction Methods and Their Implications to Point-of-Care Diagnostics. BioMed Research International Volume 2017, Article ID 9306564, 13 pages”. Solution 502 preferably includes a low conductivity eluent liquid, typically introduced during preparation of the solution, that promotes electronic addressing of nucleic acids and promotes activity of restriction enzymes in solution 502. A preferred eluent liquid includes histidine and a restriction enzyme buffer. FIG. 5B shows the dried target molecule-specific microgel deposits 190 in their dehydrated state. The introduction of solution 502 into electrophoretic array assembly 500, so that solution 502 contacts dried target molecule-specific microgel deposits 190, defines a time T0 and causes dried target molecule-specific microgel deposits 190 (FIGS. 2A & 2B) to be hydrated to their hydrated form, designated by reference numeral 170 (FIGS. 1A & 1B).

FIG. 5C illustrates immobilized target molecule-specific microgel deposits 170 in a hydrated state, as tire result of the introduction of solution 502, containing nucleic acid target molecules 503, which fills the interior of the electrophoretic array assembly 500. FIG. 5C illustrates that the hydrated target molecule-specific electrically separated microgel deposits 170 have a typical maximum height of 1.25 mm. The hydration of the target molecule-specific microgel deposits 170 to their state shown in FIG. 5C preferably takes about 10 seconds and is preferably completed at time T=T0+10 seconds.

FIG. 5D illustrates electrophoretic addressing of nucleic acids to the hydrated immobilized, mutually electrically separated target molecule-specific microgel deposits 170 in the presence of a DC electric field preferably of 10-300 Volts per centimeter. Portions of the electric field lines are indicated by reference numerals 510 and the direction of the electric field is indicated by arrows 512. It may be appreciated that the electric field lines define three-dimensional, immobilized, mutually spaced and mutually electrically separated microgel regions 520, each centered about a different target molecule-specific microgel deposit 170.

It is appreciated that addressing as well as (he various steps described hereinbelow with reference to FIGS. 5E-5I occur not only on the surface of microgel deposits 170 but also within the volume of the microgel deposits 170.

As seen in FIG. 5D, the application of the DC electric field to the three-dimensional, immobilized, mutually spaced and mutually electrically separated target molecule-specific microgel regions 520 causes rapid transport of nucleic acid target molecules 503 and nucleic acid target molecule specific RCA circular probes 320 to the target molecule-specific microgel deposits 170, which in turn facilitates specific hybridization between the nucleic acid target molecules 503 and the nucleic acid target molecule specific RCA circular probes 320. Rapid transport of nucleic acid target molecule specific RCA circular probes 320 to the target molecule-specific microgel deposits 170 also facilitates specific hybridisation between the forward primers 322, which are bound to the target molecule-specific microgel deposits 170, and the nucleic acid target molecule specific RCA circular probes 320.

The duration of the stage illustrated in FIG. 5D is between 30 seconds and 120 seconds. The stage illustrated in FIG. 5D is preferably completed at a time T=T0+[40 to 130] seconds.

Reference is now made to FIG. 5E, which illustrates a ligation stage that normally follows the addressing stage shown in FIG. 5D and preferably takes place in the presence of a DC electric field preferably of 10-300 Volts per centimeter in the same direction as the electric field in the step of FIG. 5D. The ligation stage preferably occurs in the presence of a ligation enzyme 528, for example, T-4 ligase, which is introduced into electrophoretic array assembly 500 through a solution. The duration of the ligation stage is typically 120 to 240 seconds. The ligation stage is preferably completed at T=T0+[160 to 370] seconds.

Reference is now made to FIG. 5F, which illustrates an RCA polymerization stage that normally follows the ligation stage shown in FIG. 5E and preferably occurs in the presence of a DC electric field, preferably of 10-300 Volts per centimeter in the same direction as the electric field in the step of FIG. 5E. The RCA polymerization stage preferably occurs in the presence of a Bst polymerase enzyme 529, dNTPs (not shown) and a reverse primer 324, which are introduced into electrophoretic array assembly 500 through solution, and forward primer 322, which is bound to target-molecule specific microgel deposit 170, preferably at a temperature of 65 degrees Celsius. A result of the RCA polymerization stage is generation of long RCA amplicons 540 (FIG. 5G). As seen in FIG. 5F, following binding of polymerase enzyme 529 to nucleic acid target molecule specific RCA circular probes 320, nucleic acid target molecules 503 are displaced from nucleic acid target molecule specific RCA circular probes 320, as indicated by arrows 542. The duration of the RCA polymerization stage is typically 300 to 720 seconds. The RCA polymerization stage is preferably completed at T=T0+[560 to 1090] seconds.

Reference is now made to FIG. 5G, which illustrates an amplicon stretching stage that normally occurs during the RCA polymerization stage of FIG. 5F and preferably occurs in the presence of a DC electric field, preferably of 10-300 Volts per centimeter in a direction opposite to that of the electric field in the step of FIG. 5F as indicated by an arrow 544. Stretching of amplicon 540 occurs in a direction indicated by an arrow 548. The amplicon stretching stage preferably occurs in the presence of a Bst polymerase enzyme 529, dNTPs (not shown) and reverse primer 324 at a temperature of 65 degrees Celsius. The duration of the amplicon stretching stage is typically 5 to 15 seconds. The RCA polymerization stage is preferably completed at T=T0+[565 to 1105] seconds.

It is appreciated that the stages shown in FIGS. 5P and 5G may be repeated intermittently multiple times, with multiple stages shown in FIG. 5F each being of a shorter duration than that indicated above and being separated by a stage shown in FIG. 5G.

Reference is now made to FIG. 5H, which illustrates an exponential RCA amplification stage that normally occurs during the RCA polymerization stage of FIG. 5F and the amplicon stretching stage of FIG. 5G and preferably occurs in the presence of a DC electric field, preferably of 10-300 Volts per centimeter, which is normally in an opposite direction, as indicated by arrows 512, from that of the electric field in the step of FIG. 5G but preferably includes short periods in which the polarity of the electric field is reversed. In this stage, additional amplicons 550 are generated using reverse primers 324.

The exponential RCA amplification stage preferably occurs in the presence of a Bst polymerase enzyme 529, dNTPs (not shown) and reverse primer 324 at a temperature of 65 degrees Celsius. The duration of the exponential RCA amplification stage, which occurs during the RCA polymerization stage of FIG. 5F and the amplicon stretching stage of FIG. 5G, is typically 5-15 seconds. The RCA polymerization stage of FIG. 5F, the amplicon stretching stage of FIG. 5G and the exponential RCA amplification stage of FIG. 5H, preferably are completed at T=T0+[465 to 1105] seconds.

Reference is now made to FIG. 5I, which illustrates a post-RCA polymerization addressing stage that normally occurs following the completion of RCA polymerization stage of FIG. 5F, the amplicon stretching stage of FIG. 5G and the exponential RCA amplification stage of FIG. 5H, and preferably occurs in the presence of a DC electric field, preferably of 10-300 Volts per centimeter, which is normally in the same direction as that of the electric field in the step of FIG. 5H. The post-RCA polymerization addressing stage is particularly useful for gathering amplicons 540 that ate in solution 502 at a distance from target molecule-specific microgel deposits 170 and recapturing than at the target molecule-specific microgel deposits 170, as indicated by arrows 560, and preferably concentrating the amplicons 540 at one location within each microgel region 520. The duration cf the post-RCA polymerization addressing stage is typically 10-30 seconds. The pest-RCA polymerization addressing stage is preferably completed at T=T0+[475 to 1135] seconds.

Reference is now made to FIG. 5J, which illustrates a reporting stage that normally follows the post RCA polymerization addressing stage. The reporting stage preferably occurs in the presence of a fluorescence reporter 570 complementary to amplicons 540 and 550, which is introduced to electrophoretic array assembly 500 through a solution. The duration of the reporting stage is typically 10-30 seconds. The reporting stage is preferably completed at T=T0+[585 to 1165] seconds.

Upon completion of the reporting stage and a subsequent washing stage, not shown, the detection of the presence of at least one nucleic acid target molecule, from among a multiplicity of pre-selected nucleic acid target molecules, may be carried out by conventional fluorescence detection techniques. It is thus appreciated that detection of at least one nucleic acid target molecule is preferably completed within between 8 minutes and 20 minutes of the initial supply of solution 502 to the interior of the electrophoretic array assembly 100.

It is appreciated that if preparation of solution 502 is completed within 4-5 minutes of acquisition of a sample as by taking a blood sample from a patient, detection at least one nucleic acid target molecule may be completed within 12-25 minutes from sample acquisition.

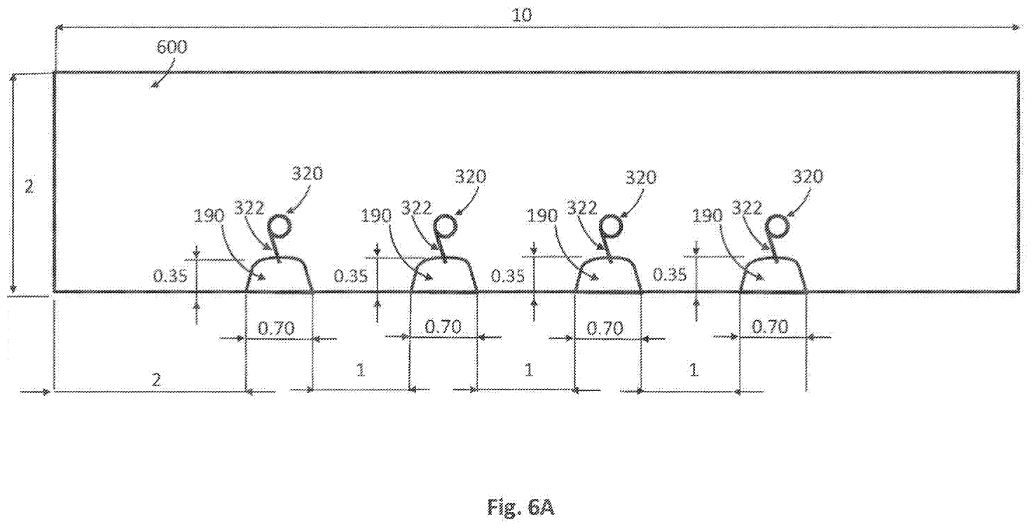

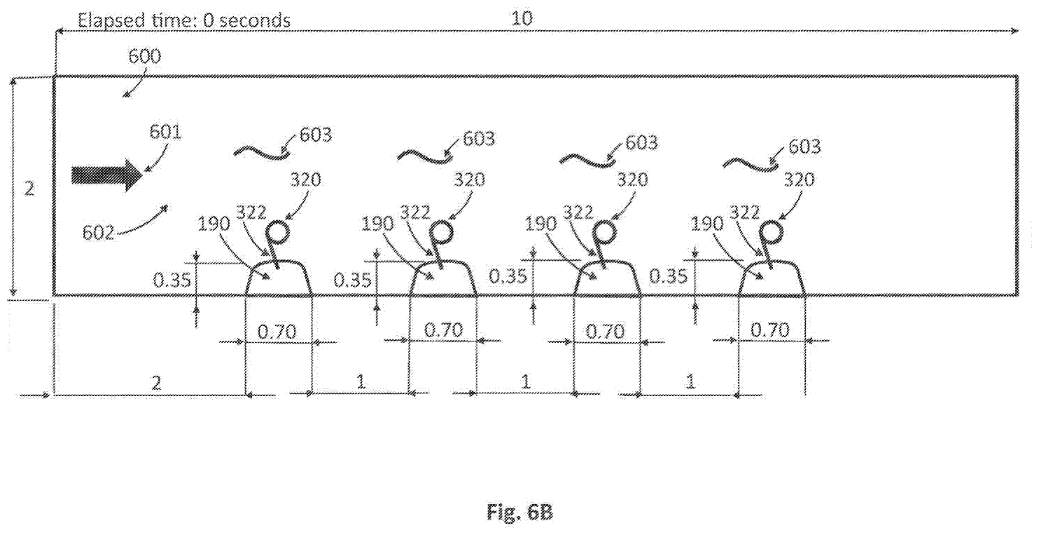

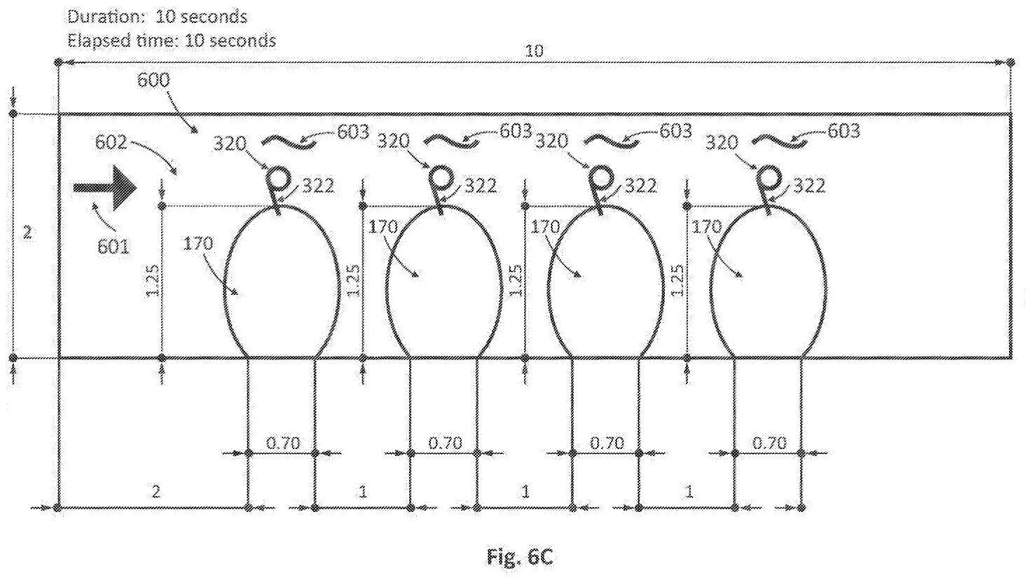

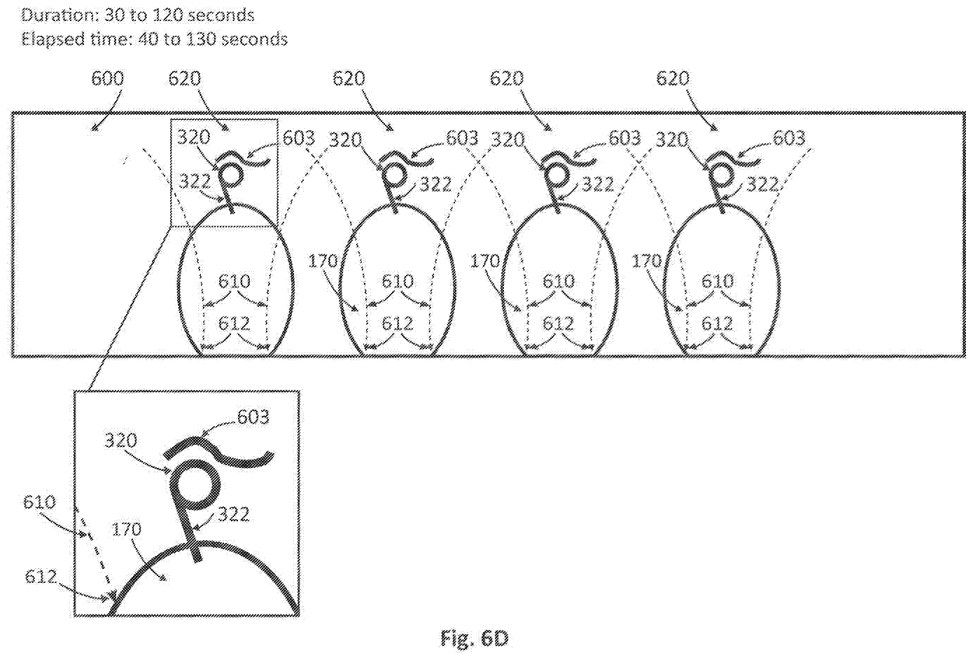

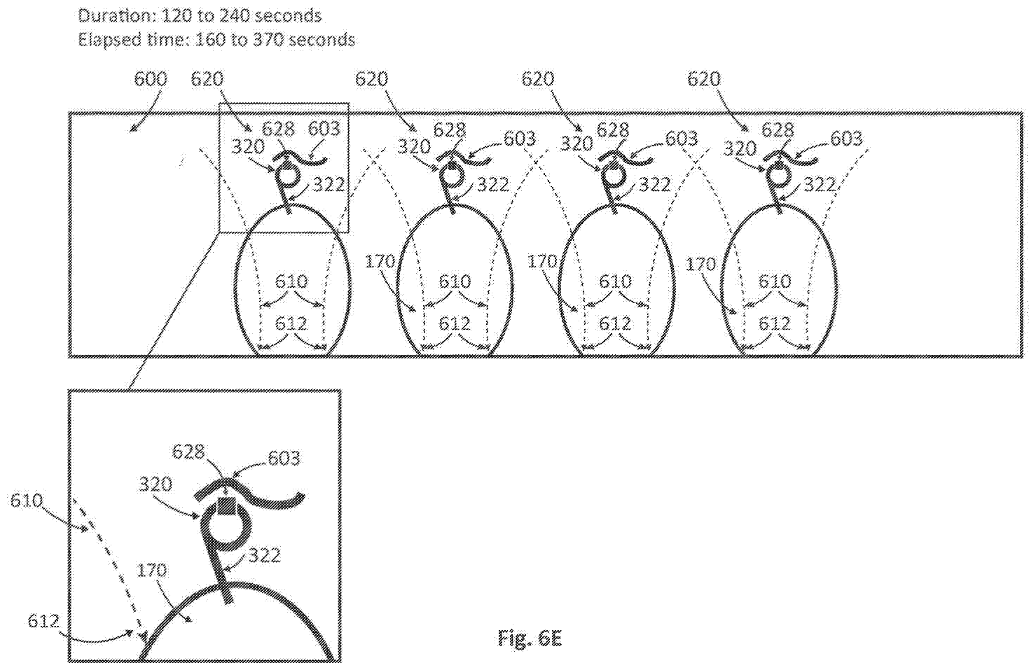

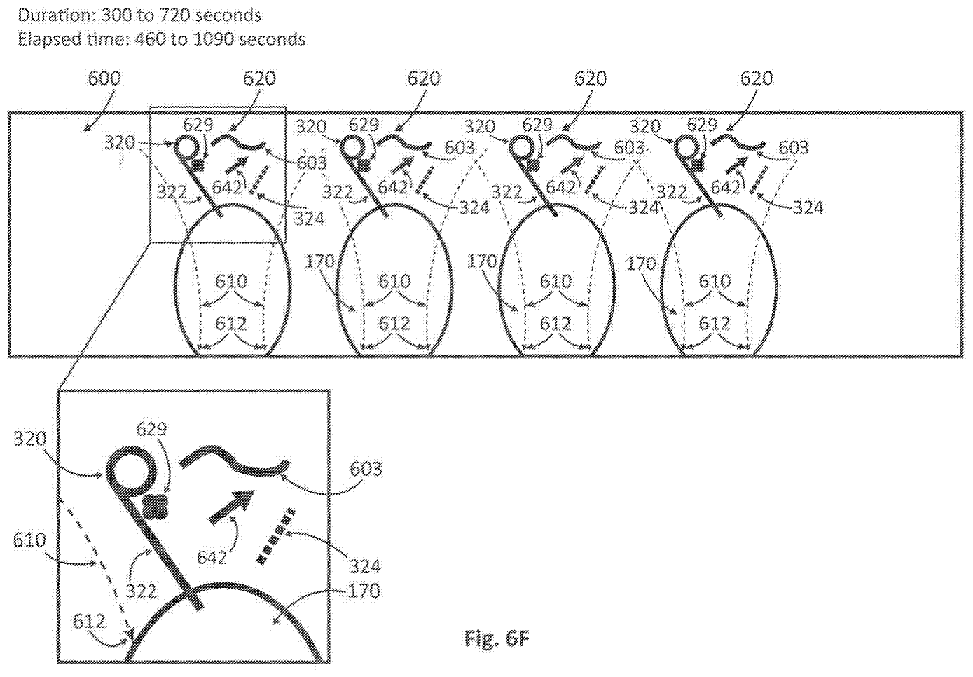

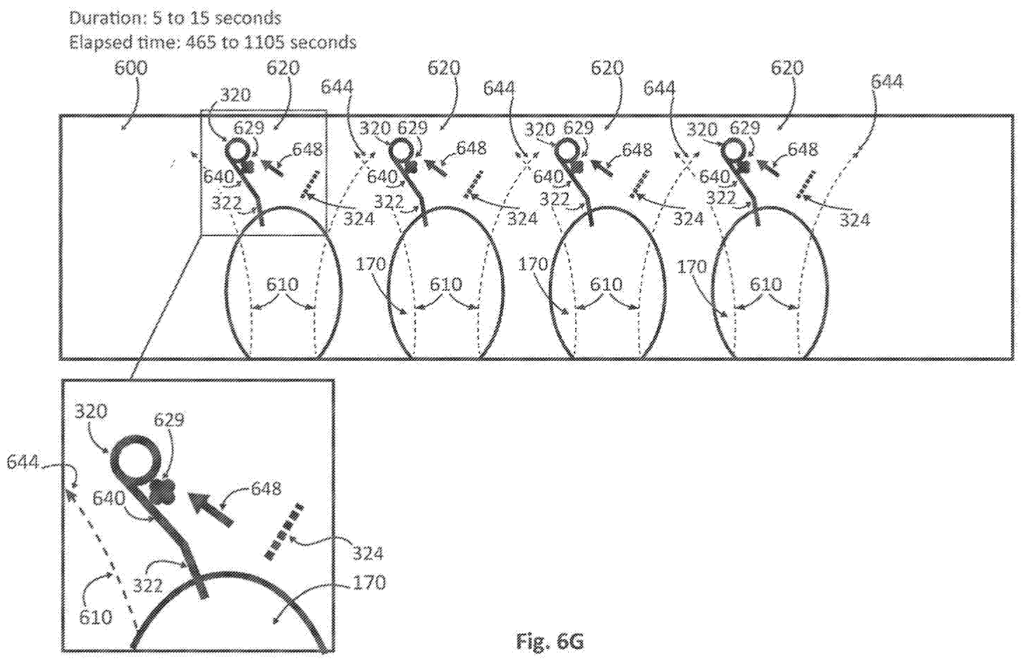

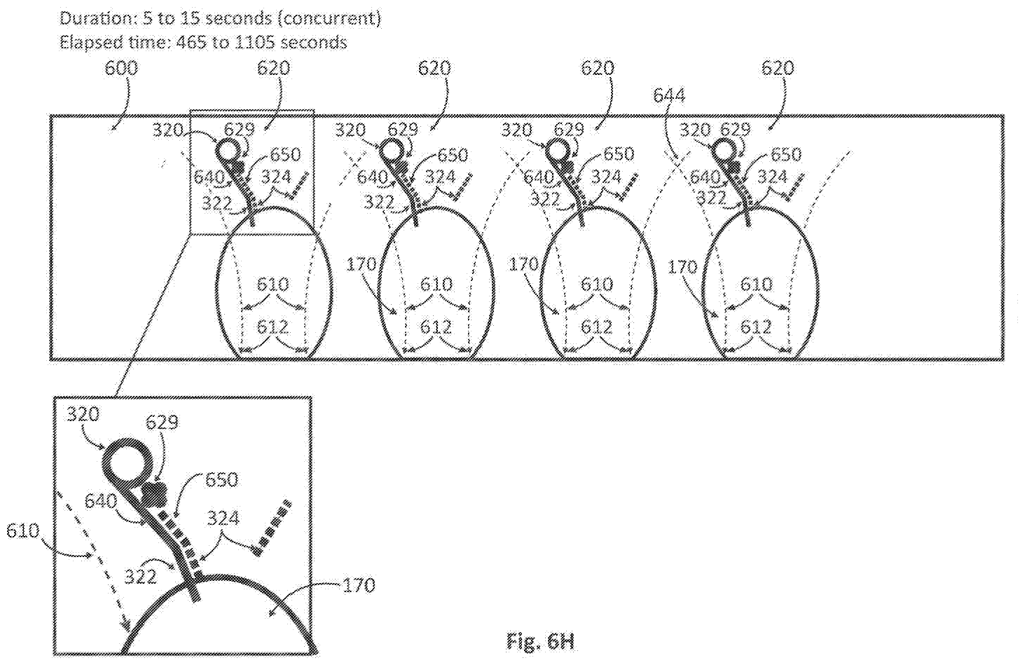

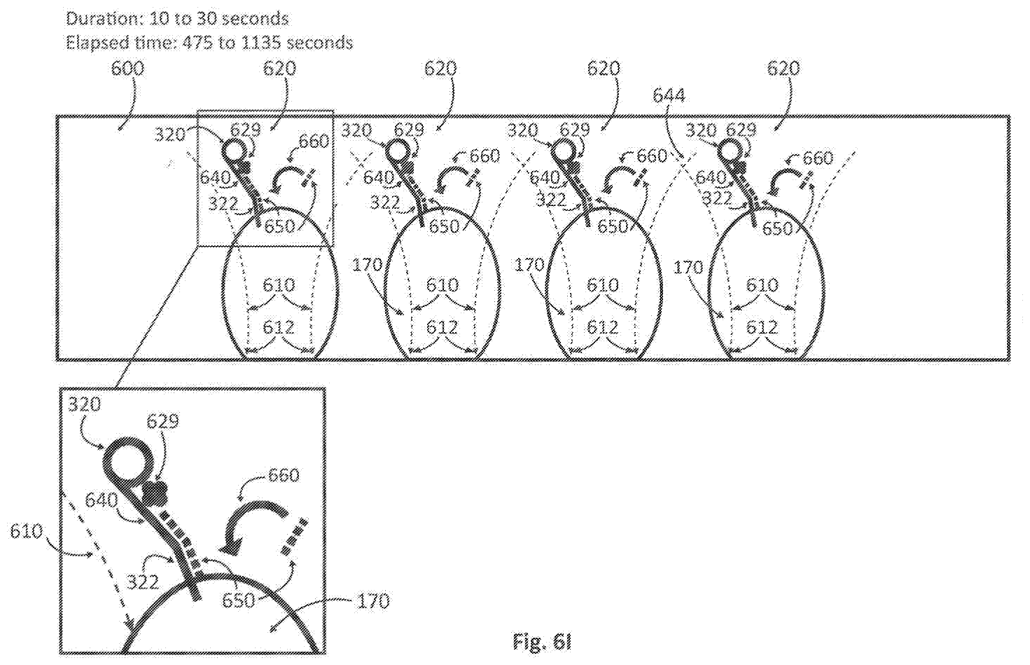

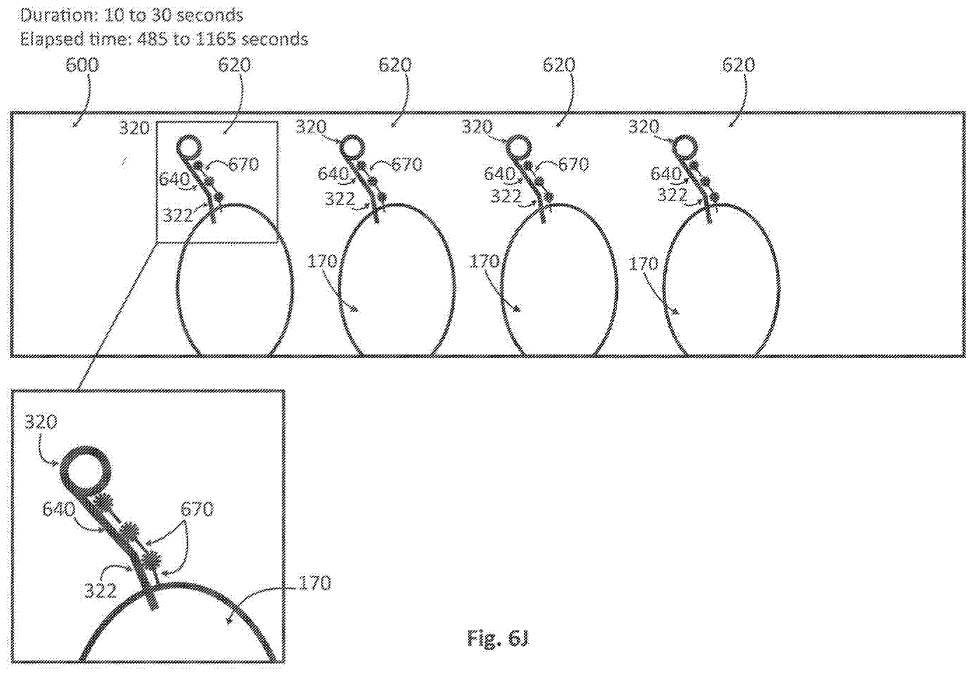

Reference is now made to FIGS. 6A-6J, which illustrate principal stages in rapid detection of the presence of at least one nucleic acid target molecule, from among a multiplicity of pre-selected nucleic acid target molecules in accordance with yet another preferred embodiment of the present invention.

The method of FIGS. 6A-6J is preferably carried out using an electrophoretic array assembly 600 in a shelf storable state, as described above with reference to FIGS. 2A & 2B, wherein forward primers 322 and nucleic acid target molecule specific RCA circular prices 320 are bound to the immobilized and mutually spaced microgel deposits 190. It is appreciated that for simplicity, the substrate 110, the peripheral wall structure 120 and the window 130 as well as the various layers constituting the electrophoretic array 160 are not specifically shown. Each of FIGS. 6A-6J is a simplified side view sectional illustration taken generally along lines 4-4 in FIG. 2A.

FIG. 6A shows an electrophoretic array assembly 600 in its dried, dehydrated, operative orientation, similar to that shown in FIG. 2A, with symbolic, not to scale, indications of various different oligonucleotide forward primers 322 and nucleic acid target molecule specific RCA circular probes 320, which are bound to corresponding different ones of the dried microgel deposits 190.

FIG. 6A indicates, in millimeter units, the interior dimensions of a preferred embodiment of the electrophoretic array assembly 600, as well as the general dimensions of the immobilized dried target molecule-specific microgel deposits 190.

It is appreciated that the method of FIGS. 6A-6J differs from the method of FIGS. 4A-4J in that instead of capture probes 400 being bound to the immobilized dried target molecule-specific microgel deposits 190 in the method of FIGS. 4A-4J, nucleic acid target molecule specific RCA circular probes 320 are specifically hybridized to forward primers 322, which are bound to the immobilized dried target molecule-specific microgel deposits 190 in the method of FIGS. 6A-6J.

FIG. 6B illustrates the introduction, symbolized by an arrow 601, of a solution 602 containing nucleic acid target molecules 603, so as to till the interior of the electrophoretic array assembly 600.

Reparation of solution 602 is not part of the present claimed invention and is carried out in accordance with conventional techniques, such as those described in “Nasir Ali, Rita de Cássia Pontello Rampazzo, Alexandre Dias Tavares Costa, and Marco Aurelio Krieger, Current Nucleic Acid Extraction Methods and Their Implications to Point-of-Care Diagnostics. BioMed Research International Volume 2017, Article ID 93065. Solution 602 preferably includes a low conductivity eluent liquid, typically introduced during preparation of the solution, that promotes electronic addressing of nucleic acids and promotes activity of restriction enzymes in solution 602. A preferred eluent liquid includes histidine and a restriction enzyme buffer. FIG. 6B shows the dried target molecule-specific microgel deposits 190 in their dehydrated state. The introduction of solution 602 into electrophoretic array assembly 100, so that solution 602 contacts dried target molecule specific microgel deposits 190, defines a time T0 and causes dried target molecule-specific microgel deposits 190 to assume their hydrated state, designated by reference numeral 170(FIGS. 1A & 1B).

FIG. 6C illustrates immobilized target molecule-specific microgel deposits 170 in a hydrated state, as the result of the introduction of solution 602, containing nucleic acid target molecules 603, which fills the interior of the electrophoretic array assembly 600. FIG. 6C illustrates that the hydrated target molecule-specific electrically separated microgel deposits 170 have a typical maximum height of 1.25 mm. The hydration of the target molecule-specific microgel deposits 190 to their state shown in FIG. 6C preferably takes about 10 seconds and is preferably completed at time T=T0+10 seconds.

FIG. 6D illustrates electrophoretic addressing of nucleic acids to the hydrated immobilized, mutually electrically separated target molecule-specific microgel deposits 170 in the presence of a DC electric field preferably of 10-300 Volts per centimeter. Portions of the electric field lines are indicated by reference numerals 610 and the direction of the electric field is indicated by arrows 612. It may be appreciated that the electric field lines define three dimensional, immobilized, mutually spaced and mutually electrically separated microgel regions 620, each centered about a different target molecule-specific microgel deposit 170.

It is appreciated that addressing as well as the various steps described herein below with reference to FIGS. 6F-6J occur tun only on the surface of microgel deposits 170 but also within the volume of the microgel deposits 170.

As seen in FIG. 6D. the application of the DC electric field to the three-dimensional, immobilized, mutually spaced and mutually electrically separated target molecule-specific microgel regions 620 causes rapid transport of nucleic acid target molecules 603 to the target molecule-specific microgel deposits 170, which in turn facilitates specific hybridization between the nucleic acid target molecules 603 and the nucleic acid target molecule specific RCA circular probes 320, which RCA circular probes 320 are bound to forward primers 322, which forward primers 322 are bound lo hydrated target molecule specific electrically separated microgel deposits 170. The duration of the stage illustrated in FIG. 6D is between 30 seconds and 120 seconds. The stage illustrated in FIG. 6D is preferably completed at a time T=T0+[40 to 130] seconds.

Reference is now made to FIG. 6F. which illustrates a ligation stage that normally follows the addressing stage shown in FIG. 6D and preferably takes place in the presence of a DC electric field, preferably of 10-300 Volts per centimeter in the same direction as the electric field in the step of FIG. 6D. The ligation stage preferably occurs in the presence of a ligation enzyme 628, for example, T-4 ligase, which is introduced into electrophoretic array assembly 600 through a solution. The duration of the ligation stage is typically 120 lo 240 seconds. The ligation stage is preferably completed at T=T0+[160 to 370] seconds.

Reference is now made to FIG. 6F, which illustrates an RCA polymerization stage that normally follows the ligation stage shown in FIG. 6E and preferably occurs in the presence of a DC electric field, preferably of 10-300 Volts per centimeter in the same direction as the electric field in the step of FIG. 6E. The RCA polymerization stage preferably occurs in the presence of a Bst polymerase enzyme 629, dNTPs (not shown) and a reverse primer 324, which are introduced into electrophoretic array assembly 600 through solution, and forward primer 322, which is bound to target-molecule specific microgel deposit 170, preferably at a temperature of 65 degrees Celsius. A result of the RCA polymerization stage is generation of long RCA amplicons 640 (FIG. 6G). As seen in FIG. 6F, following binding of polymerase enzyme 629 to nucleic acid target molecule specific RCA circular probes 320, nucleic acid target molecules 603 are displaced from nucleic acid target molecule specific RCA circular probes 320, as indicated by arrows 642. The duration of the RCA polymerization stage is typically 300 to 720 seconds. The RCA polymerization stage is preferably completed at T=T0+[460 to 1090] seconds.

Reference is now made to FIG. 6G, which illustrates an amplicon stretching stage that normally occurs during the RCA polymerization stage of FIG. 6F and preferably occurs in the presence of a DC electric field, preferably of 10-300 Volts per centimeter in a direction opposite to that of the electric field in the step of FIG. 6F as indicated by an arrow 644. Stretching of amplicon 640 occurs in a direction indicated by an arrow 648. The amplicon stretching stage preferably occurs in the presence of a Bst polymerase enzyme 629, dNTPs (not shown) and reverse primer 324 at a temperature of 65 degrees Celsius. The duration of the amplicon stretching stage is typically 5 to 15 seconds. The RCA polymerization stage if preferably completed at T=T0+[465 to 1105] seconds.

It is appreciated that the stages shown in FIGS. 6F and 6G may be repeated intermittently multiple times, with multiple stages shown in FIG. 6F each being of a shorter duration than that indicated above and being separated by a stage shown in FIG. 6G.

Reference is now made to FIG. 6H, which illustrates an exponential RCA amplification stage that normally occurs during the RCA polymerization stage of FIG. 6F and the amplicon stretching stage of FIG. 6G and preferably occurs in the presence of a DC electric field, preferably of 10-300 Volts per centimeter, which is normally in an opposite direction from that of the electric field in the step of FIG. 6G but preferably includes short periods in which the polarity of the electric field is reversed. In this stage, additional amplicons 650 are generated using reverse primers 324.

The exponential RCA amplification stage preferably occurs in the presence of a Bst polymerase enzyme 629, dNTPs (not shown) and reverse primer 324 at a temperature of 65 degrees Celsius. The duration of the exponential RCA amplification stage, which occurs during the RCA polymerization stage of FIG. 6F and the amplicon stretching stage of FIG. 6G, is typically 5-15 seconds. The RCA polymerization stage of FIG. 6F, the amplicon stretching stage of FIG. 6G and the exponential RCA amplification stage of FIG. 6H, preferably are completed at T=T0+[465 to 1105] seconds.

Reference is now made to FIG. 6I, which illustrates a post-RCA polymerization addressing stage that normally occurs following the completion of RCA polymerization stage of FIG. 6F, the amplicon stretching stage of FIG. 6G and the exponential RCA amplification stage of FIG. 6H, and preferably occurs in the presence of a DC electric field, preferably of 10-300 Volts per centimeter, which is normally in the same direction as that of the electric field in the step of FIG. 6H. The post-RCA polymerization addressing stage is particularly useful for gathering amplicons 640 that are in solution 602 at a distance from target molecule-specific microgel deposits 170 and recapturing them at the target molecule-specific microgel deposits 170, as indicated by arrows 660, and preferably concentrating the amplicons 640 at one location within each microgel region 620. The duration of the post-RCA polymerization addressing stage is typically 10-30 seconds. The post-RCA polymerization addressing stage is preferably completed at T=T0+[475 to 1135] seconds.

Reference is now made to FIG. 6J, which illustrates a reporting stage that normally follows the post-RCA polymerization addressing stage. The reporting stage preferably occurs in the presence of a fluorescence reporter 670 complementary to amplicons 640 and 650, which is introduced to electrophoretic array assembly 600 through a solution. The duration of the reporting stage is typically 10-30 seconds. The reporting stage is preferably completed at T=T0+[485 to 1165] seconds.

Upon completion of the reporting stage and a subsequent washing stage, not shown, the detection of the presence of at least one nucleic acid target molecule, from among a multiplicity of pre-selected nucleic acid target molecules, may be carried out by conventional fluorescence detection techniques. It is thus appreciated that detection of at least one nucleic acid target molecule Is preferably completed within between 8 minutes and 20 minutes of the initial supply of solution 602 to the interior of the electrophoretic array assembly 600.

It is appreciated that if preparation of solution 602 is completed within 4-5 minutes of acquisition of a sample as by taking a blood sample from a patient, detection at least one nucleic acid target molecule may be completed within 12-25 minutes from sample acquisition.

Reference is now made to FIGS. 7A-7J, which illustrate principal stages in rapid detection of the presence of at least one nucleic acid target molecule, from among a multiplicity of pre-selected nucleic acid target molecules in accordance with still another preferred embodiment of the present invention. It is appreciated that for simplicity, the substrate 110, the peripheral wall structure 120 and the window 130 as well as the various layers constituting the electrophoretic array 160 are not specifically shown. Each of FIGS. 7A-7J is a simplified side view sectional illustration taken generally along lines 4-4 in FIG. 2A.

FIG. 7A shows an electrophoretic array assembly 700 in its dried, dehydrated, operative orientation, similar to that shown in FIG. 2A, with symbolic indications of various different oligonucleotide forward primers 322, nucleic acid target molecule specific RCA circular probes 320 and reverse printers 324 which are bound to corresponding different ones of the dried microgel deposits 190. FIG. 7A indicates, in millimeter units, the interior dimensions of a preferred embodiment of the electrophoretic array assembly 700, as well as the general dimensions of the immobilized dried target molecule-specific microgel deposits 190.

It is appreciated that the method of FIGS. 7A-7J differs from the method of FIGS. 4A-4J in that instead of capture probes 400 being bound to the immobilized dried target molecule specific microgel deposits 190 in the method of FIGS. 4A-4J, nucleic acid target molecule specific RCA circular probes 320 are specifically hybridized to forward primers 322, which are bound to immobilized dried target molecule-specific microgel deposits 190, and reverse primers 324 are also bound to immmobilized dried target molecule-specific microgel deposits 190 in the method of FIGS. 7A-7J.

FIG. 7B illustrates the introduction, symbolized by an arrow 701, of a solution 702 containing nucleic acid target molecules 703, so as to fill the interior of the electrophoretic array assembly 700.

Preparation of solution 702 is not part of the present claimed invention and is earned out in accordance with conventional techniques, such as those described in “Nasir Ali, Rita de Cássia Pontello Rampazzo, Alexandre Dias Tavares Costa, and Marco Aurelio Krieger, Current Nucleic Acid Extraction Methods and Their Implications to Point-of-Care Diagnostics. BioMed Research International Volume 2017, Article ID 93065. Solution 702 preferably includes a low conductivity eluent liquid, typically introduced during preparation of the solution, that promotes electronic addressing of nucleic acids and promotes activity of restriction enzymes in solution 702. A preferred eluent liquid includes histidine and a restriction enzyme buffer. FIG. 7B shows the dried target molecule-specific microgel deposits 190 in their dehydrated state. The introduction of solution 702 into electrophoretic array assembly 700, so that solution 702 contacts dried target molecule specific microgel deposits 190, defines a time T0 and causes dried target molecule-specific microgel deposits 190 to assume their hydrated state, designated by reference numeral 170 (FIGS. 1A & 1B).

FIG. 7C illustrates the immobilized target molecule-specific microgel deposits in a hydrated state, as indicated by reference numerals 170 (FIGS. 1A & 1B) as the result of the introduction of solution 702, containing nucleic acid target molecules 703, which fills the interior of the electrophoretic array assembly 700. FIG. 7C illustrates thru the hydrated target molecule-specific electrically separated microgel deposits 170 have a typical maximum height of 1.25 mm. The hydration of the target molecule-specific microgel deposits 170 to their hydrated state shown in FIG. 7C preferably takes about 10 seconds and is preferably completed at time T=T0+10 seconds.

FIG. 7D illustrates electrophoretic addressing of nucleic acid target molecules 703 to the hydrated immobilized, mutually electrically separated microgel deposits 170 in the presence of a DC electric field preferably of 10-300 Volts per centimeter. Portions of the electric field lines are indicated by reference numerals 710 and the direction of the electric field is indicated by arrows 712. It may be appreciated that the electric field lines define three-dimensional, immobilized, mutually spaced and mutually electrically separated microgel regions 720, each centered about a different target molecule-specific microgel deposit 170.

It is appreciated that addressing as well as the various steps described hereinbelow with reference to FIGS. 7E-7J occur not only on the surface of microgel deposits 170 but also within the volume of the microgel deposits 170. As seen in FIG. 7D, the application of the DC electric field to the three-dimensional, immobilized, mutually spaced and mutually electrically separated target molecule-specific microgel regions 720 causes rapid transport of nucleic acid target molecules 703 to the target molecule-specific microgel deposits 170, which in turn facilitates specific hybridization between the nucleic acid target molecules 703 and the nucleic acid target molecule specific RCA circular probes 320, which RCA circular probes 320 are bound to forward primers 322, which forward primers 322 are bound to the target molecule-specific microgel deposits 170.

The duration of the stage illustrated in FIG. 7D is between 30 seconds and 120 seconds. The stage illustrated in FIG. 7D is preferably completed at a time T=T0+[40 to 130] seconds.

It is appreciated that an optional removing stage (not shown) may be added following the addressing stage shown in FIG. 7D, prior to the stages described below in reference to 7E-7J, and preferably takes place in the presence of a DC electric field, preferably of 10 -300 Volts per centimeter in the opposite direction as the electric field in the step of FIG. 7D. The removing stage preferably is operative to remove non-specific target molecules which had hybridized to RCA circular probes 320.

Reference is now made to FIG. 7E, which illustrates a ligation stage that normally follows the addressing stage shown in FIG. 7D and preferably takes place in the presence of a DC electric field, preferably of 10-300 Volts per centimeter in the same direction as the electric field in the step of FIG. 7D. The ligation stage preferably occurs in the presence of a ligation enzyme 728, for example, T-4 ligase, which is introduced into electrophoretic array assembly 700 through a solution. The duration of the ligation stage is typically 120 to 240 seconds. The ligation stage is preferably completed at T=T0+[160 to 370] seconds.

Reference Is now made to FIG. 7F, which illustrates an RCA polymerization stage that normally follows the ligation stage shown in FIG. 7E and preferably occurs in the presence of a DC electric field, preferably of 10-300 Volts per centimeter in the same direction as the electric Held in the step of FIG. 7E. The RCA polymerization stage preferably occurs in the presence of a Bst polymerase enzyme 729 and dNTPs (not shown), which are introduced into electrophoretic array assembly 700 through a solution, and forward primer 322 and reverse primer 324, which are bound to the target molecule-specific microgel deposit 170, preferably at a temperature of 65 degrees Celsius. A result of the RCA polymerization stage is generation of long RCA amplicons 740 (FIG. 7G). As seen in FIG. 7F, following binding of polymerase enzyme 729 to nucleic acid target molecule specific RCA circular probes 320, nucleic acid target molecules 703 are displaced from nucleic acid target molecule specific RCA circular probes 320, as indicated by arrows 742. The duration of the RCA polymerization stage is typically 300 to 720 seconds. The RCA polymerization stage is preferably completed at T=T0+[460 to 1090] seconds.

Reference is now made to FIG. 7G, which illustrates an amplicon stretching and compressing stage that normally occurs during the RCA polymerization stage of FIG. 7F and preferably occurs in the presence of a DC electric field, preferably of 10-300 Volts per centimeter in directions both the same and opposite to that of the electric field in the step of FIG. 7F as indicated by an arrow 744. Stretching of amplicon 740 occurs in a direction indicated by an arrow 748. Compressing typically occurs in a direction opposite to that shown by arrow 748. Amplicon stretching and compressing enhances hybridization of reverse primers 324, which are bound to the target molecule-specific microgel deposits 170, to amplicon 740. The amplicon stretching and compressing stage preferably occurs in the presence of a Bst polymerase enzyme 729, dNTPs (not shown) at a temperature of 65 degrees Celsius. The duration of the amplicon stretching stage is typically 5 to 15 seconds. The RCA polymerization stage is preferably completed at T=T0+[465 to 1105] seconds.

It is appreciated that the stages shown in FIGS. 7F and 7G may be repealed intermittently multiple times, with the stages shown in FIG. 7F being each of a shorter duration than that indicated above and being separated by a stage shown in FIG. 7G.

Reference is now made to FIG. 7H, which illustrates an exponential RCA amplification stage that normally occurs during the RCA polymerization stage of FIG. 7F and the amplicon compressing stretching stage of FIG. 7G and preferably occurs in the presence of a DC electric field, preferably of 10-300 Volts per centimeter, which is normally in an opposite direction from that of the electric field in the step of FIG. 7G, but preferably includes short periods in which the polarity of the electric field is reversed. In this stage, additional amplicons 750 are generated using reverse primers 324.

The exponential RCA amplification stage preferably occurs in the presence of a Bst polymerase enzyme 729, dNTPs (not shown) and reverse primer 324 at a temperature of 65 degrees Celsius. The duration of the exponential RCA amplification stage, which occurs during the RCA polymerization stage of FIG. 7F and the amplicon stretching stage of FIG. 7G, is typically 5-15 seconds. The RCA polymerization stage of FIG. 7F, the amplicon stretching stage of FIG. 7G and the exponential RCA amplification stage of FIG. 7H, preferably are completed at T=T0+[465 to 1105] seconds.

Reference is now made to FIG. 7I, which illustrates a post-RCA amplicon concentrating stage that normally occurs following the completion of RCA polymerization stage of FIG. 7F, the amplicon stretching and compressing stage of FIG. 7G and the exponential RCA amplification stage of FIG. 7H, and preferably occurs in the presence of a DC electric field, preferably of 10-300 Volts per centimeter, which is normally in the same direction as that of the electric field in the step of FIG. 7H. The amplicon concentrating stage is particularly useful for concentrating amplicons 740 and 750, as indicated by arrows 760, at one location within each microgel region 720. The duration of the amplicon concentrating stage is typically 10-30 seconds. The post-RCA polymerization addressing stage is preferably completed at T=T0+[475 to 1135]seconds.

Reference is now made to FIG. 7J, which illustrates a reporting stage that normally follows the post-RCA polymerization addressing. The reporting stage preferably occurs in the presence of a fluorescence reporter 770 complementary to amplicons 740 and 750, winch is introduced to electrophoretic array assembly 700 through a solution. The duration of the reporting stage is typically 10-30 seconds. The reporting stage is preferably completed at T=T0+[485 to 1165] seconds.

Upon completion of the reporting stage and a subsequent washing stage, not shown, the detection of the presence of at least one nucleic acid target molecule, from among a multiplicity of pre selected nucleic acid target molecules, may be carried out by conventional fluorescence defection techniques. It is thus appreciated that detection of at least one nucleic acid target molecule is preferably completed within between approximately 8 minutes and 20 minutes of the initial supply of solution 702 to the interior of the electrophoretic array assembly 100.

It is appreciated that if preparation of solution 701 is completed within 4-5 minutes of acquisition of a sample as by taking a blood sample from a patient, detection at least one nucleic acid target molecule may be completed within 12-25 minutes from sample acquisition.

### EXAMPLES

### Example 1

Detection of Meningitis Pathogens Employing the Method of FIGS. 7A-7J and using a Synthetic DNA Target Molecule Representing Neisseria meningitidis

An electrophoretic array assembly similar to electrophoretic array assembly 700 (FIG. 7A) containing 100 immobilized dried target molecule-specific microgel deposits 190 having a base diameter of 0.45 mm and a height of approximately 0.2 mm-0.3 mm was provided. 48 immobilized dried target molecule-specific microgel deposits 190 were spotted with RCA circular probes 320 pre-hybridized to forward primers 322 and with reverse primers 324 specific to nine different pathogen DNA targets each relevant to detection of meningitis, including inter alia Neisseria meningitidis. Accordingly, the 48 spotted immobilized dried target molecule-specific microgel deposits 190 were target molecule-specific as follows:
Deposit 1—Specific to Neisseria meningitidis
Deposit 2—Specific to Neisseria meningitidis
Deposit 3—Specific to Neisseria meningitidis
Deposit 4.—Specific to Escherichia coli
Deposit 5.—Specific to Escherichia coli
Deposit 6.—Specific to Escherichia coli
Deposit 7—Specific to Neisseria meningitidis
Deposit 8—Specific to Neisseria meningitidis
Deposit 9—Specific to Neisseria meningitidis
Deposit 10. Specific to Enterovirus
Deposit 11. Specific to Enterovirus
Deposit 12—Specific to Neisseria meningitidis
Deposit 13—Specific to Neisseria meningitidis
Deposit 14—Specific to Neisseria meningitidis
Deposit 15. Group B Streptococcus
Deposit 16. Group B Streptococcus
Deposit 17. Group B Streptococcus
Deposit 18—Specific to Neisseria meningitidis
Deposit 19—Specific to Neisseria meningitidis
Deposit 20—Specific to Neisseria meningitidis
Deposit 21—Specific to Haemophilus influenzae
Deposit 22—Specific to Haemophilus influenzae
Deposit 23—Specific to Haemophilus influenzae
Deposit 24—Specific to Neisseria meningitidis
Deposit 25—Specific to Neisseria meningitidis
Deposit 26—Specific to Neisseria meningitidis
Deposit 27—Specific to Human herpes virus
Deposit 28—Specific to Human herpes virus
Deposit 29—Specific to Human herpes virus
Deposit 30—Specific to Human herpes virus
Deposit 31—Specific to Neisseria meningitidis
Deposit 32—Specific to Neisseria meningitidis
Deposit 33—Specific to Neisseria meningitidis
Deposit 34—Specific to Human parechovirus
Deposit 35—Specific to Human parechovirus
Deposit 36—Specific to Human parechovirus
Deposit 37—Specific to Neisseria meningitidis
Deposit 38—Specific to Neisseria meningitidis
Deposit 39—Specific to Neisseria meningitidis
Deposit 40—Specific to Lysteria monocytogenes
Deposit 41—Specific to Lysteria monocytogenes
Deposit 42—Specific to Lysteria monocytogenes
Deposit 43—Specific to Neisseria meningitidis
Deposit 44—Specific to Neisseria meningitidis
Deposit 45—Specific to Neisseria meningitidis
Deposit 46. Specific to Varicella zoster
Deposit 47. Specific to Varicella zoster
Deposit 48. Specific to Varicella zoster

A solution 702 containing nucleic acid target molecules 703 (100 nM concentration) representing Neisseria meningitidis was supplied to the interior volume of the electrophoretic array, at a time defined as T0. The solution 702 also included a low conductivity buffer supporting rapid DNA transport and hybridization to the RCA probes deposited on the microgels.

Supplying solution 702 caused dried target molecule-specific microgel deposits 190 to assume their hydrated state, designated by reference numeral 170, after a duration of 10 seconds. (FIGS. 7B-7C)
At time T=T0+10 seconds, a constant current of 1.6 mA was applied across the working and counter electrode contacts 260 and 250 respectively, resulting in voltages of 4.5 V, yielding an electric field applied across the electrophoretic array of 12.5 V per cm and producing electrophoretic addressing (FIG. 7D). The duration of the electrophoretic addressing was 40 seconds.

At time T=T0+50 seconds, a ligation reaction solution including ligation reaction enzyme T4 ligase (Blunt T/A, from New England Biolabs) was supplied to the interior volume of the electrophoretic array, replacing solution 702, for a duration of approximately 180 seconds. (FIG. 7E)

At time T=T0+230 seconds, a polymerase solution containing list polymerase enzyme 729 and dNTPs (from New England Biolabs) was supplied to the interior volume of the electrophoretic array, replacing the ligation reaction solution, for a duration of approximately 720 seconds. (FIG. 7P).

At time T=T0+950 seconds, a constant current of 1.6 mA was applied across the working and counter electrode contacts 260 and 250 respectively, resulting in voltages of 4.5 V, yielding an electric field applied across the electrophoretic array of 12.5 V per cm and providing recapture of RCA amplicons from the polymerase solution. The duration of this step was approximately 20 seconds. (FIG. 7I)

At time T=T0+970 seconds, a red reporter solution containing fluorescently labeled oligonucleotides (Alexa 647 from Integrated Device Technology, Inc., San Jose, Calif.) was supplied to the interior volume of the electrophoretic array, replacing the polymerase solution for a duration of approximately 30 seconds. (FIG. 7J). Following washing out of the red reporter solution, a fluorescence image of the electrophoretic array assembly 700 was taken via window 130 and the presence of nucleic acid target molecules 703 representing Neisseria meningitidis was detected at the following ones of immobilized dried target molecule-specific microgel deposits 170: 1, 2, 3, 7, 8, 9, 13, 14, 15, 19, 20, 21, 25, 26, 27, 31, 32, 33, 37, 38, 39, 43, 44, and 45. The presence of nucleic acid target molecules 703 representing Neisseria meningitidis was not detected at the following ones of immobilized dried target molecule-specific microgel deposits 170: 4, 5, 6, 10, 11, 12, 16, 17, 18, 22, 23, 24, 28, 29, 30, 34, 35, 36, 40, 41, 42, 46, 47 and 48.

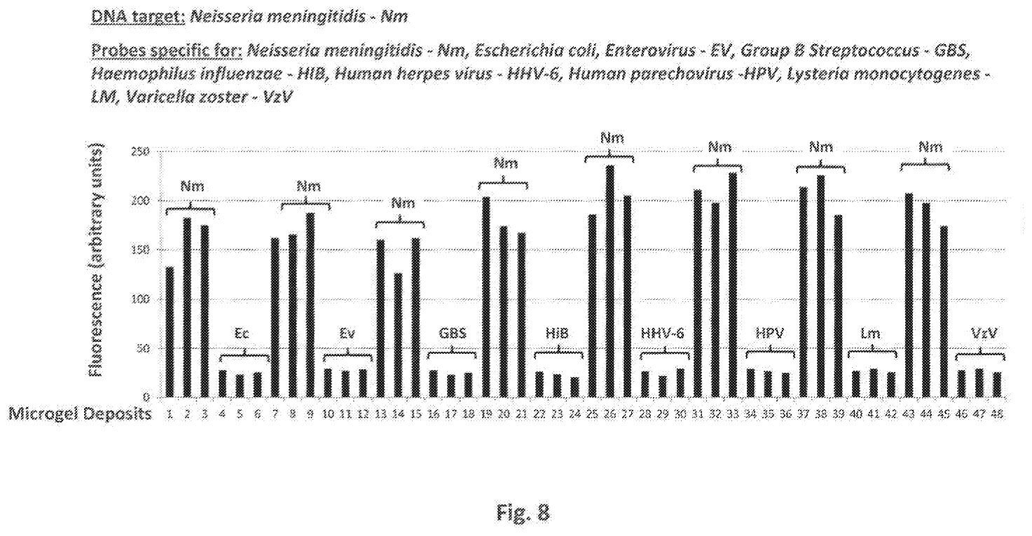

The detection results are summarized in FIG. 8. It is noted that the average ratio of intensities of the fluorescence signal obtained from deposits 1, 2, 3, 7, 8, 9, 13, 14, 15, 19, 20, 21, 25, 26, 27, 31, 32, 33, 37, 38, 39, 43, 44, and 45 to the fluorescence signal obtained from deposits 4, 5, 6, 10, 11, 12, 16, 17, 18, 22, 23, 24, 28, 29, 30, 34, 35, 36, 40, 41, 42, 46, 47 and 48 was approximately 8.5.

### Example 2

Detection of Meningitis Pathogens Employing the Method of FIGS. 7A-7J and using a Genomic DNA Target Molecule Extracted from Neisseria meningitidis Pathogen Spiked into Cerebrospinal Fluid Clinical Sample

An electrophoretic array assembly similar to electrophoretic array assembly 700 (FIG. 7A) containing 100 immobilized dried target molecule specific microgel deposits 190 having a base diameter of 0.45 mm and a height of approximately 0.2 mm-0.3 mm was provided. 21 immobilized dried target molecule-specific microgel deposits 190 were spotted with RCA circular probes 320 pro-hybridized to forward printers 322 and with reverse primers 324 specific to nine different pathogen DNA targets each relevant to detection of meningitis, including inter alia Neisseria meningitidis. Accordingly, the 21 spotted immobilized dried target molecule-specific microgel deposits 190 were target molecule-specific as follows:
Deposit 1. Specific to Escherichia coli
Deposit 2. Specific to Escherichia coli
Deposit 3—Specific to Neisseria meningitidis
Deposit 4—Specific to Neisseria meningitidis
Deposit 5—Specific to Neisseria meningitidis
Deposit 6. Specific to Enterovirus
Deposit 7. Specific to Enterovirus
Deposit 8—Specific to Neisseria meningitidis
Deposit 9—Specific to Neisseria meningitidis
Deposit 10—Specific to Neisseria meningitidis
Deposit 11. Group B Streptococcus
Deposit 12. Group B Streptococcus
Deposit 13—Specific to Haemophilus influenzae
Deposit 14—Specific to Haemophilus influenzae
Deposit 15—Specific to Neisseria meningitidis
Deposit 16—Specific to Neisseria meningitidis
Deposit 17—Specific to Neisseria meningitidis
Deposit 18—Specific to Lysteria monocytogenes
Deposit 19—Specific to Lysteria monocytogenes
Deposit 20. Specific to Varicella zoster
Deposit 21. Specific to Varicella zoster

A clinical sample of cerebrospinal fluid (CSF) was spiked with Neisseria meningitides pathogen, and genomic DNA extraction performed using a common magnetic bead-based DNA extraction method. The input concentration of DNA target in cerebrospinal fluid was determined by a reference real-time PCR method that yielded Neisseria meningitides pathogen concentration in clinical sample of cerebrospinal fluid of 720 copies of DNA per microliter of CSF, was input.

A solution 702, prepared from the spiked clinical sample, was supplied to the interior volume of the electrophoretic array, at a time defined as T0. The solution 702 also included a low conductivity buffer supporting rapid DNA transport and hybridisation to the RCA probes deposited on the microgels.

Supplying solution 702 caused dried target molecule-specific microgel deposits 190 to assume their hydrated state, designated by reference numeral 170, after a duration of 10 seconds. (FIGS. 7B-7C)

At time T=T0+10 seconds, a constant current of 1.6 mA was applied across the working and counter electrode contacts 260 and 250 respectively, resulting in voltages of 4.5 V, yielding an electric field applied across the electrophoretic array of 12.5 V per cm and producing electrophoretic addressing (FIG. 7D). The duration of the electrophoretic addressing was 40 seconds.

At time T=T0+50 seconds, a reverse polarity electric field was applied by applying a constant current of negative 1.6 mA across the working and counter electrode contacts 260 and 250 respectively, resulting in voltages of 4.5 V, yielding an electric field applied across the electrophoretic array of 12.5 V per cm and enhancing removal of nonspecifically bound DNA targets. The duration of the electrophoretic addressing was 10 seconds. (FIG. 7G)

At time T=T0+60 seconds, a ligation reaction solution including ligation reaction enzyme T4 ligase (Blunt T/A, from New England Biolabs) was supplied to the interior volume of the electrophoretic array, replacing solution 702, for a duration of approximately 180 seconds. (FIG. 7E)

At time T=T0+240 seconds, a polymerase solution containing list polymerase enzyme 729 and dNTPs (from New England Biolabs) was supplied to the interior volume of the electrophoretic array, replacing the ligation reaction solution, for a duration of approximately 720 seconds. (FIG. 7F).

At time T=T0+960 seconds, a constant current of 1.6 mA was applied across the working and counter electrode contacts 260 and 250 respectively, resulting in voltages of 4.5 V, yielding an electric field applied across the electrophoretic array of 12.5 V per cm and providing recapture of RCA amplicons from the polymerase solution. The duration of this step was approximately 20 seconds. (FIG. 7I)

At lime T=T0+980 seconds, a red reporter solution containing fluorescently labeled oligonucleotides (Alexa 647 from Integrated Device Technology, Inc., San Jose, Calif.) was supplied to the interior volume of the electrophoretic array, replacing the polymerase solution for a duration of approximately 30 seconds. (FIG. 7J). Following washing out of the red reporter solution, a fluorescence image of the electrophoretic array assembly 700 was taken via window 130 and the presence of nucleic acid target molecules 703 representing Neisseria meningitidis was detected at the following ones of immobilized dried target molecule-specific microgel deposits 170: 3, 4, 5, 8, 9, 10, 15, 16, and 17. The presence of nucleic acid target molecules 703 representing Neisseria meningitidis was not detected at the following ones of immobilized dried target molecule-specific microgel deposits 170: 1, 2, 6, 7, 11, 12, 13, 14, 18, 19, 20, and 21.

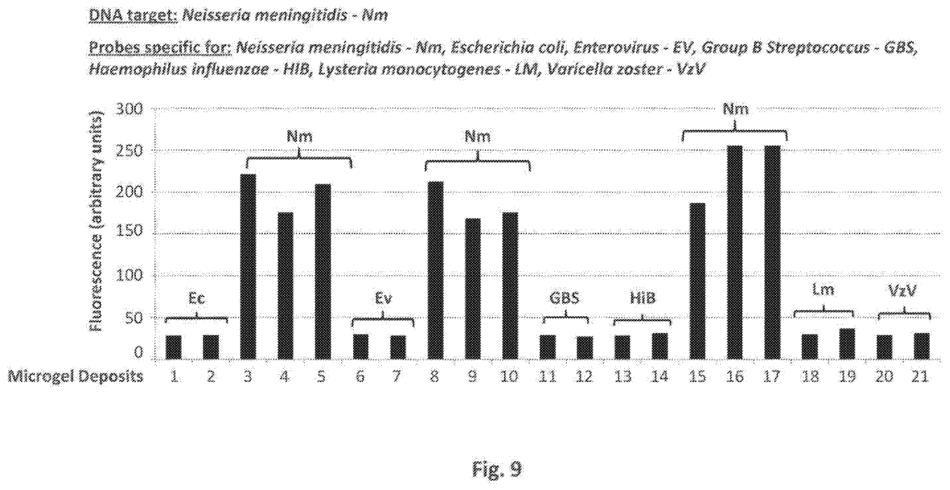

The detection results are summarized in FIG. 9. It is noted that the average ratio of intensities of the fluorescence signal obtained from deposits 3, 4, 5, 8, 9, 10, 15, 16, and 17 to the the fluorescence signal obtained from deposits 1, 2, 6, 7, 11, 12, 13, 14, 18, 19, 20, and 21 was approximately 4.3.

It will be appreciated by persons skilled in the art that the present invention is not limited to what has been specifically described and shown herein but also includes combinations and sub-combinations of features described herein and modifications thereof which are not in the prior art.

1. For use in a method of rapidly detecting the presence of at least one nucleic acid target molecule, from among a multiplicity of pre-selected nucleic acid target molecules, in a solution, a room-temperature shelf-storable electrophoretic array comprising:
a multiplicity of immobilized, mutually spaced and mutually electrically separated microgel deposits, each of said multiplicity of immobilized, mutually spaced and mutually electrically separated microgel deposits containing materials suitable for performing rolling circle amplification and binding of at least one of said multiplicity of pre-selected nucleic acid target molecules, each of said microgel deposits containing at least the following elements pre-anchored therein:
an RCA probe specific to of at least one of said multiplicity of pre-selected nucleic acid target molecules; and
at least one primer.

2. An electrophoretic array according to claim 1, wherein said microgel deposits are dehydrated and are rehydratable when exposed to a solution containing at least one nucleic acid target molecule.

3. An electrophoretic array according to claim 1, wherein said at least one primer includes at least one forward primer and at least one reverse primer.

4. An electrophoretic array according to claim 1, wherein said RCA probe is pre-hybridized to said at least one primer.

5. An electrophoretic array according to claim 1, wherein each of said microgel deposits when hydrated has a generally hemispherical shaped configuration.

6. An electrophoretic array according to claim 1, wherein:
said multiplicity of immobilized, mutually spaced and mutually electrically separated microgel deposits define a corresponding multiplicity of immobilized, mutually spaced and mutually electrically separated microgel regions; and
said electrophoretic array is employed in carrying out a method comprising:
introducing said solution to each of said multiplicity of immobilized, mutually spaced and mutually electrically separated microgel regions;
performing rolling circle amplification at least generally simultaneously at each of said multiplicity of immobilized, mutually spaced and mutually electrically separated microgel regions, while applying electric fields thereto during various stages of said rolling circle amplification; and
detecting the presence of at least one of said multiplicity of pre-selected nucleic acid target molecules at at least one corresponding one of said immobilized, mutually spaced and mutually electrically separated microgel regions,
wherein said detecting occurs within a short time period of said introducing, said short time period being less than 30 minutes.

7. An electrophoretic array according to claim 6, wherein said detecting comprises optical detection.

8. An electrophoretic array according to claim 6, wherein said detecting comprises fluorescence detection.

9. An electrophoretic array according to claim 6, wherein said applying electric fields thereto occurs during at least two different stages in said rolling circle amplification.

10. An electrophoretic array according to claim 6, wherein said electric fields are at least generally the same at each of said immobilized, mutually spaced and mutually electrically separated microgel regions.

11. An electrophoretic array according to claim 6, wherein said detecting occurs within a time duration of less than 20 minutes.

12. An electrophoretic array according to claim 6, wherein said detecting occurs within a time duration of less than 15 minutes.

13. An electrophoretic array according to claim 6, wherein said applying electric fields during said rolling circle amplification comprises at least one of the following:
applying an electric field to said immobilized, mutually spaced and mutually electrically separated microgel regions for driving nucleic acid target molecules in said solution to said microgel deposits;
applying an electric field to said immobilized, mutually spaced and mutually electrically separated microgel regions for driving nucleic acid target molecule-RCA probe hybridization products in said solution to said microgel deposits;
applying an electric field to said immobilized, mutually spaced and mutually electrically separated microgel regions for recapturing RCA amplicons that drift away from said microgel deposits;
applying an electric field to said immobilized, mutually spaced and mutually electrically separated microgel regions for driving RCA probes into said microgel deposits for hybridization with at least one of capture probes and primers already bound to said microgel deposits;
applying an electric field to said immobilized, mutually spaced and mutually electrically separated microgel regions for removing undesired molecules from said microgel regions;
applying an electric field to said immobilized, mutually spaced and mutually electrically separated microgel regions for stretching RCA amplicons that are bound to said microgel deposits;
applying an electric field to said immobilized, mutually spaced and mutually electrically separated microgel regions for compressing RCA amplicons that are bound to said microgel deposits;
applying an electric field to said immobilized, mutually spaced and mutually electrically separated microgel regions for stirring RCA reagents in the vicinity of RCA amplicons that are bound to said microgel deposits;
applying an electric field to said immobilized, mutually spaced and mutually electrically separated microgel regions for enhancing the speed of enzyme activity in RCA; and
applying electric field of sequentially reversing polarity to said immobilized, mutually spaced and mutually electrically separated microgel regions for enhancing stringency of binding of RCA amplicons to said microgel deposits.

14. An electrophoretic array according to claim 6, wherein said applying electric fields during said rolling circle amplification comprises at least two of the following:
applying an electric field to said immobilized, mutually spaced and mutually electrically separated microgel regions for driving nucleic acid target molecules in said solution to said microgel deposits;
applying an electric field to said immobilized, mutually spaced and mutually electrically separated microgel regions for driving nucleic acid target molecule-RCA probe hybridization products in said solution to said microgel deposits;
applying an electric field to said immobilized, mutually spaced and mutually electrically separated microgel regions for recapturing RCA amplicons that drift away from said microgel deposits;
applying an electric field to said immobilized, mutually spaced and mutually electrically separated microgel regions for driving RCA probes into said microgel deposits for hybridization with at least one of capture probes and primers already bound to said microgel deposits;
applying an electric field to said immobilized, mutually spaced and mutually electrically separated microgel regions for removing undesired molecules from said microgel regions;
applying an electric field to said immobilized, mutually spaced and mutually electrically separated microgel regions for stretching RCA amplicons that are bound to said microgel deposits;
applying an electric field to said immobilized, mutually spaced and mutually electrically separated microgel regions for compressing RCA amplicons that are bound to said microgel deposits;
applying an electric field to said immobilized, mutually spaced and mutually electrically separated microgel regions for stirring RCA reagents in the vicinity of RCA amplicons that are bound to said microgel deposits;
applying an electric field to said immobilized, mutually spaced and mutually electrically separated microgel regions for enhancing the speed of enzyme activity in RCA; and
applying electric field of sequentially reversing polarity to said immobilized, mutually spaced and mutually electrically separated microgel regions for enhancing stringency of binding of RCA amplicons to said microgel deposits.

15. An electrophoretic array according to claim 6, wherein said applying electric fields during said rolling circle amplification comprises at least three of the following:
applying an electric field to said immobilized, mutually spaced and mutually electrically separated microgel regions for driving nucleic acid target molecules in said solution to said microgel deposits;
applying an electric field to said immobilized, mutually spaced and mutually electrically separated microgel regions for driving nucleic acid target molecule-RCA probe hybridization products in said solution to said microgel deposits;
applying an electric field to said immobilized, mutually spaced and mutually electrically separated microgel regions for recapturing RCA amplicons that drift away from said microgel deposits;
applying an electric field to said immobilized, mutually spaced and mutually electrically separated microgel regions for driving RCA probes into said microgel deposits for hybridization with at least one of capture probes and primers already bound to said microgel deposits;
applying an electric field to said immobilized, mutually spaced and mutually electrically separated microgel regions for removing undesired molecules from said microgel regions;
applying an electric field to said immobilized, mutually spaced and mutually electrically separated microgel regions for stretching RCA amplicons that are bound to said microgel deposits;
applying an electric field to said immobilized, mutually spaced and mutually electrically separated microgel regions for compressing RCA amplicons that are bound to said microgel deposits;
applying an electric field to said immobilized, mutually spaced and mutually electrically separated microgel regions for stirring RCA reagents in the vicinity of RCA amplicons that are bound to said microgel deposits;
applying an electric field to said immobilized, mutually spaced and mutually electrically separated microgel regions for enhancing the speed of enzyme activity in RCA; and
applying electric field of sequentially reversing polarity to said immobilized, mutually spaced and mutually electrically separated microgel regions for enhancing stringency of binding of RCA amplicons to said microgel deposits.

16. An electrophoretic array according to claim 6, wherein said applying electric fields during said rolling circle amplification comprises at least four of the following:
applying an electric field to said immobilized, mutually spaced and mutually electrically separated microgel regions for driving nucleic acid target molecules in said solution to said microgel deposits;
applying an electric field to said immobilized, mutually spaced and mutually electrically separated microgel regions for driving nucleic acid target molecule-RCA probe hybridization products in said solution to said microgel deposits;
applying an electric field to said immobilized, mutually spaced and mutually electrically separated microgel regions for recapturing RCA amplicons that drift away from said microgel deposits;
applying an electric field to said immobilized, mutually spaced and mutually electrically separated microgel regions for driving RCA probes into said microgel deposits for hybridization with at least one of capture probes and primers already bound to said microgel deposits;
applying an electric field to said immobilized, mutually spaced and mutually electrically separated microgel regions for removing undesired molecules from said microgel regions;
applying an electric field to said immobilized, mutually spaced and mutually electrically separated microgel regions for stretching RCA amplicons that are bound to said microgel deposits;
applying an electric field to said immobilized, mutually spaced and mutually electrically separated microgel regions for compressing RCA amplicons that are bound to said microgel deposits;
applying an electric field to said immobilized, mutually spaced and mutually electrically separated microgel regions for stirring RCA reagents in the vicinity of RCA amplicons that are bound to said microgel deposits;
applying an electric field to said immobilized, mutually spaced and mutually electrically separated microgel regions for enhancing the speed of enzyme activity in RCA; and
applying electric field of sequentially reversing polarity to said immobilized, mutually spaced and mutually electrically separated microgel regions for enhancing stringency of binding of RCA amplicons to said microgel deposits.

17. A method of rapidly detecting the presence of at least one nucleic acid target molecule, from among a multiplicity of pre-selected nucleic acid target molecules, in a solution, the method comprising:
introducing said solution to at least a multiplicity of immobilized, mutually spaced and mutually electrically separated microgel regions on an electrophoretic array, each of said multiplicity of immobilized, mutually spaced and mutually electrically separated microgel regions containing a microgel deposit containing materials suitable for binding of a different one of said multiplicity of pre-selected nucleic acid target molecules and performing rolling circle amplification;
performing rolling circle amplification at least generally simultaneously at said immobilized, mutually spaced and mutually electrically separated microgel regions, while applying electric fields thereto during various stages of said rolling circle amplification; and
detecting the presence of at least one of said multiplicity of pre-selected nucleic acid target molecules at least one corresponding one of said immobilized, mutually spaced and mutually electrically separated microgel regions,
wherein said detecting occurs within a short time period of said introducing, said short time period being less than 30 minutes.

18. A method of rapidly detecting the presence of at least one nucleic acid target molecule according to claim 17 and wherein said detecting comprises optical detection.

19. A method of rapidly detecting the presence of at least one nucleic acid target molecule according to claim 17 and wherein said detecting comprises fluorescence detection.

20. A method of rapidly detecting the presence of at least one nucleic acid target molecule according to claim 17, wherein said applying electric fields thereto occurs during at least two different stages in said rolling circle amplification.

21. A method of rapidly detecting the presence of at least one nucleic acid target molecule according to claim 17, wherein said electric fields are at least generally the same at each of said immobilized, mutually spaced and mutually electrically separated microgel regions.

22. A method of rapidly detecting the presence of at least one nucleic acid target molecule according to claim 17, wherein said detecting occurs within a time duration of less than 20 minutes.

23. A method of rapidly detecting the presence of at least one nucleic acid target molecule according to claim 17, wherein said detecting occurs within a time duration of less than 15 minutes.

24. A method of rapidly detecting the presence of at least one nucleic acid target molecule according to claim 17, wherein said applying electric fields during said rolling circle amplification comprises at least one of the following:
applying an electric field to said immobilized, mutually spaced and mutually electrically separated microgel regions for driving nucleic acid target molecules in said solution to said microgel deposits;
applying an electric field to said immobilized, mutually spaced and mutually electrically separated microgel regions for driving nucleic acid target molecule-RCA probe hybridization products in said solution to said microgel deposits;
applying an electric field to said immobilized, mutually spaced and mutually electrically separated microgel regions for recapturing RCA amplicons that drift away from said microgel deposits;
applying an electric field to said immobilized, mutually spaced and mutually electrically separated microgel regions for driving RCA probes into said microgel deposits for hybridization with at least one of capture probes and primers already bound to said microgel deposits;
applying an electric field to said immobilized, mutually spaced and mutually electrically separated microgel regions for removing undesired molecules from said microgel regions;
applying an electric field to said immobilized, mutually spaced and mutually electrically separated microgel regions for stretching RCA amplicons that are bound to said microgel deposits;
applying an electric field to said immobilized, mutually spaced and mutually electrically separated microgel regions for compressing RCA amplicons that are bound to said microgel deposits;
applying an electric field to said immobilized, mutually spaced and mutually electrically separated microgel regions for stirring RCA reagents in the vicinity of RCA amplicons that are bound to said microgel deposits;
applying an electric field to said immobilized, mutually spaced and mutually electrically separated microgel regions for enhancing the speed of enzyme activity in RCA; and
applying electric field of sequentially reversing polarity to said immobilized, mutually spaced and mutually electrically separated microgel regions for enhancing stringency of binding of RCA amplicons to said microgel deposits.

25. A method of rapidly detecting the presence of at least one nucleic acid target molecule according to claim 17, wherein said applying electric fields during said rolling circle amplification comprises at least two of the following:
applying an electric field to said immobilized, mutually spaced and mutually electrically separated microgel regions for driving nucleic acid target molecules in said solution to said microgel deposits;
applying an electric field to said immobilized, mutually spaced and mutually electrically separated microgel regions for driving nucleic acid target molecule-RCA probe hybridization products in said solution to said microgel deposits;
applying an electric field to said immobilized, mutually spaced and mutually electrically separated microgel regions for recapturing RCA amplicons that drift away from said microgel deposits;
applying an electric field to said immobilized, mutually spaced and mutually electrically separated microgel regions for driving RCA probes into said microgel deposits for hybridization with at least one of capture probes and primers already bound to said microgel deposits;
applying an electric field to said immobilized, mutually spaced and mutually electrically separated microgel regions for removing undesired molecules from said microgel regions;
applying an electric field to said immobilized, mutually spaced and mutually electrically separated microgel regions for stretching RCA amplicons that are bound to said microgel deposits;
applying an electric field to said immobilized, mutually spaced and mutually electrically separated microgel regions for compressing RCA amplicons that are bound to said microgel deposits;
applying an electric field to said immobilized, mutually spaced and mutually electrically separated microgel regions for stirring RCA reagents in the vicinity of RCA amplicons that are bound to said microgel deposits;
applying an electric field to said immobilized, mutually spaced and mutually electrically separated microgel regions for enhancing the speed of enzyme activity in RCA; and
applying electric field of sequentially reversing polarity to said immobilized, mutually spaced and mutually electrically separated microgel regions for enhancing stringency of binding of RCA amplicons to said microgel deposits.

26. A method of rapidly detecting the presence of at least one nucleic acid target molecule according to claim 17, wherein said applying electric fields during said rolling circle amplification comprises at least three of the following:
applying an electric field to said immobilized, mutually spaced and mutually electrically separated microgel regions for driving nucleic acid target molecules in said solution to said microgel deposits;
applying an electric field to said immobilized, mutually spaced and mutually electrically separated microgel regions for driving nucleic acid target molecule-RCA probe hybridization products in said solution to said microgel deposits;
applying an electric field to said immobilized, mutually spaced and mutually electrically separated microgel regions for recapturing RCA amplicons that drift away from said microgel deposits;
applying an electric field to said immobilized, mutually spaced and mutually electrically separated microgel regions for driving RCA probes into said microgel deposits for hybridization with at least one of capture probes and primers already bound to said microgel deposits;
applying an electric field to said immobilized, mutually spaced and mutually electrically separated microgel regions for removing undesired molecules from said microgel regions;
applying an electric field to said immobilized, mutually spaced and mutually electrically separated microgel regions for stretching RCA amplicons that are bound to said microgel deposits;
applying an electric field to said immobilized, mutually spaced and mutually electrically separated microgel regions for compressing RCA amplicons that are bound to said microgel deposits;
applying an electric field to said immobilized, mutually spaced and mutually electrically separated microgel regions for stirring RCA reagents in the vicinity of RCA amplicons that are bound to said microgel deposits;
applying an electric field to said immobilized, mutually spaced and mutually electrically separated microgel regions for enhancing the speed of enzyme activity in RCA; and
applying electric field of sequentially reversing polarity to said immobilized, mutually spaced and mutually electrically separated microgel regions for enhancing stringency of binding of RCA amplicons to said microgel deposits.

27. A method of rapidly detecting the presence of at least one nucleic acid target molecule according to claim 17, wherein said applying electric fields during said rolling circle amplification comprises at least four of the following:
applying an electric field to said immobilized, mutually spaced and mutually electrically separated microgel regions for driving nucleic acid target molecules in said solution to said microgel deposits;
applying an electric field to said immobilized, mutually spaced and mutually electrically separated microgel regions for driving nucleic acid target molecule-RCA probe hybridization products in said solution to said microgel deposits;
applying an electric field to said immobilized, mutually spaced and mutually electrically separated microgel regions for recapturing RCA amplicons that drift away from said microgel deposits;
applying an electric field to said immobilized, mutually spaced and mutually electrically separated microgel regions for driving RCA probes into said microgel deposits for hybridization with at least one of capture probes and primers already bound to said microgel deposits;
applying an electric field to said immobilized, mutually spaced and mutually electrically separated microgel regions for removing undesired molecules from said microgel regions;
applying an electric field to said immobilized, mutually spaced and mutually electrically separated microgel regions for stretching RCA amplicons that are bound to said microgel deposits;
applying an electric field to said immobilized, mutually spaced and mutually electrically separated microgel regions for compressing RCA amplicons that are bound to said microgel deposits;
applying an electric field to said immobilized, mutually spaced and mutually electrically separated microgel regions for stirring RCA reagents in the vicinity of RCA amplicons that are bound to said microgel deposits;
applying an electric field to said immobilized, mutually spaced and mutually electrically separated microgel regions for enhancing the speed of enzyme activity in RCA; and
applying electric field of sequentially reversing polarity to said immobilized, mutually spaced and mutually electrically separated microgel regions for enhancing stringency of binding of RCA amplicons to said microgel deposits.

28. A method of rapidly detecting the presence of at least one nucleic acid target molecule according to claim 17, wherein said electrophoretic array comprises a room-temperature shelf-storable electrophoretic array.

29. A method of rapidly detecting the presence of at least one nucleic acid target molecule according to claim 17, wherein said electrophoretic array comprises:
a multiplicity of immobilized, mutually spaced and mutually electrically separated microgel deposits, each of said multiplicity of immobilized, mutually spaced and mutually electrically separated microgel deposits containing materials suitable for performing rolling circle amplification and binding of at least one of said multiplicity of pre-selected nucleic acid target molecules,
each of said microgel deposits containing at least the following elements pre-anchored therein:
an RCA probe specific to of at least one of said multiplicity of pre-selected nucleic acid target molecules; and
at least one primer.

30. A method of rapidly detecting the presence of at least one nucleic acid target molecule according to claim 29, wherein said microgel deposits are dehydrated and are rehydratable when exposed to a solution containing at least one nucleic acid target molecule.

31. A method of rapidly detecting the presence of at least one nucleic acid target molecule according to claim 29, wherein said at least one primer includes at least one forward primer and at least one reverse primer.

32. A method of rapidly detecting the presence of at least one nucleic acid target molecule according to claim 29 and wherein said RCA probe is pre-hybridized to said at least one primer.

33. A method of rapidly detecting the presence of at least one nucleic acid target molecule according to claim 29 and wherein each of said microgel deposits when hydrated has a generally hemispherical shaped configuration.
